SEQUENCE LISTING (18 sequences)

SEQ ID NO: 1 - DNA, 369 bp, SARS-CoV
atgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctactctagtatttttaatactttgcttcaccattaagagaaagacagaatga

SEQ ID NO: 2 - protein, 122 aa, SARS-CoV
MKIILFLTLIVFTSCELYHYQECVRGTTVLLKEPCPSGTYEGNSPFHPLADNKFALTCTSTHFAFACADGTRHTYQLRARSVSPKLFIRQEEVQQELYSPLFLIVATLVFLILCFTIKRKTE

SEQ ID NO: 3 - DNA, 25 bp, SARS-CoV
cgggatccaccatgggaatgaaaat

SEQ ID NO: 4 - DNA, 21 bp, SARS-CoV
ccgctcgagtcattctgtctt

SEQ ID NO: 5 - DNA, 24 bp, SARS-CoV
cgggatccatggagctatatcact

SEQ ID NO: 6 - DNA, 36 bp, SARS-CoV
gtatttacattgttgctgctacttcactatcaggag

SEQ ID NO: 7 - DNA, 38 bp, SARS-CoV
cctgatagtgaagtagcagcaacaatgtaaatacaatc

SEQ ID NO: 8 - DNA, 28 bp, SARS-CoV
ccgctcgagtcattctgtctctctcaat

SEQ ID NO: 9 - DNA, 20 bp, SARS-CoV
ccgctcgagaattctgtctt

SEQ ID NO: 10 - DNA, 24 bp, SARS-CoV
ccgctcgagtcactcttgttgaac

SEQ ID NO: 11 - DNA, 22 bp, SARS-CoV
ccgctcgagaaggtgaagcaaa

SEQ ID NO: 12 - DNA, 22 bp, SARS-CoV
ccgctcgagaagcaaagtatta

SEQ ID NO: 13 - DNA, 24 bp, SARS-CoV
ccgctcgagtcaggtgaagcaaag

SEQ ID NO: 14 - DNA, 26 bp, SARS-CoV
ccgctcgagtcattttggtgaaactg

SEQ ID NO: 15 - DNA, 20 bp, SARS-CoV
ttgaactggtgaaactgatc

SEQ ID NO: 16 - DNA, 21 bp, SARS-CoV
tcaccagttcaacaagagctc

SEQ ID NO: 17 - DNA, 465 bp, SARS-CoV
atgatgccaactactttgtctgctggcacacacataactatggctactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaa

SEQ ID NO: 18 - protein, 154 aa, SARS-CoV
MMPTTLSAGTHITMATVYHITVSQIQLSLLKVTAFQHQNSKKTTKLVVILRIGTQVLKTMSLYMAISPKFTTSLSLHKLLQTLVLKMLHSSSLTSLLKTHRMCKYTQSTALQELLIQQWIQFMMSRRRLLACLCKHKKVSTNLCTHSFRKKQVR

(19) United States
(12) Patent Application Publication
(10) Pub. No.: US 2006/0292178 A1
(43) Pub. Date: Dec. 28, 2006

(54) Proteins encoded by the severe acute respiratory syndrome (SARS) coronavirus and a role in apoptosis

(75) Inventors: Wanjin Hong, Parc Palais (SG); Seng Gee Lim, Singapore (SG); Yee Joo Tan, Singapore (SG); Burtram Clinton Fielding, Proteos (SG); Shuo Shen, Proteos (SG); Hoe Peng Timothy Tan, Proteos (SG)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Agency for Science, Technology and Research, Centros (SG)

(21) Appl. No.: 11/197,722

(22) Filed: Aug. 4, 2005

Related U.S. Application Data
(60) Provisional application No. 60/598,683, filed on Aug. 4, 2004.

(51) Int. Cl. A61K 39/215; C12Q 1/70; C07H 21/04; C12N 15/86; C07K 14/165

(52) U.S. Cl. 424/221.1; 435/5; 435/69.3; 435/456; 435/235.1; 435/325; 530/350; 536/23.72

(57) ABSTRACT

Compositions related to expressed severe acute respiratory syndrome (SARS) coronavirus group-specific polypeptides are provided. Further provided are methods and compositions related to the use of these polypeptides, in particular use for inducing apoptosis in cells, diagnosing SARS infection and screening for modulator and inhibitor compounds of such polypeptides and in turn the virus itself, and in the treatment and/or prophylaxis of disease caused by SARS infection.

### CROSS REFERENCE TO RELATED APPLICATION

This application claims the benefit of U.S. Provisional Application Ser. No. 60/598,683, filed Aug. 4, 2004, the contents of which are herein incorporated by reference in their entirety.

### TECHNICAL FIELD

The present invention relates to expressed severe acute respiratory syndrome (SARS) coronavirus group-specific polypeptides. The present invention also relates to uses of these polypeptides, in particular use for inducing apoptosis in cells, diagnosing SARS infection and screening for modulator and inhibitor compounds of such polypeptides and in turn the virus itself, and in the treatment and/or prophylaxis of disease caused by SARS infection.

### BACKGROUND OF THE INVENTION

An outbreak of atypical pneumonia, severe acute respiratory syndrome (SARS) is thought to have originated from Guang-dong Province, Republic of China in late 2002. The mortality rate of individuals suffering from SARS can be as high as 15% (1), depending on the age group analyzed. A coronavirus (CoV) has recently been shown to fulfill all of Koch's postulates as the primary aetiological agent of SARS, including outcomes of monkey trials (5).

The SARS coronavirus (SARS-CoV) genome is ˜30 kb in length and contains 14 potential open reading frames (ORFs). These encode the replicase gene 1a/1b and the 4 structural proteins (spike (S), envelope (E), membrane (M) and nucleocapsid (N)) as well as 9 viral proteins, varying in length from 39 to 274 amino acids, with no homologue in other coronaviruses. The present invention illustrates that at least three of these viral proteins, termed as 3a, 3b and 7a, are expressed in SARS-CoV infected cells.

A question of interest is the nature of the SARS pathogenesis. Induction of apoptosis in infected cells can contribute directly to the viral pathogenesis while inhibition of apoptosis can prevent premature death of the infected cells, allowing the virus to replicate to a high titer or allowing the establishment of a persistent infection. The present invention further indicates that at least SARS coronavirus group-specific polypeptide 7a of SARS-CoV can induce apoptosis when over-expressed, and that the expression of 7a during infection is an underlying mechanism for the pathogenesis of SARS-CoV infection.

### SUMMARY OF THE INVENTION

According to a first aspect of the present invention there is provided an isolated expressed SARS coronavirus group-specific polypeptide 7a.

The expressed SARS coronavirus group-specific polypeptide may be encoded by the nucleic acid sequence of SEQ ID NO:1. The expressed SARS coronavirus group-specific polypeptide may have the amino acid sequence as set forth in SEQ ID NO: 2 or the amino acid sequence as set forth in SEQ ID NO: 2 including one or more conservative amino acid substitutions.

The expressed SARS coronavirus group-specific polypeptide as set forth in SEQ ID NO: 2 is also known as viral protein 7a. The viral protein 7a is also capable of inducing apoptosis, and this may be achieved via a caspase dependent pathway. The viral protein 7a may be induced in tumrogenic or continuous cell-lines derived from different organs.

In a second aspect, the present invention provides a vector comprising a nucleic acid sequence encoding the SARS coronavirus group-specific polypeptide of the first aspect of the invention.

In a third aspect, the present invention provides a recombinant host cell comprising the nucleic acid encoding the SARS coronavirus group-specific polypeptide in accordance with the first aspect of the invention or the vector in accordance with the second aspect of the invention.

In a fourth aspect, the present invention provides a recombinant host cell capable of expressing the SARS coronavirus group-specific polypeptide of the first aspect of the invention.

In a fifth aspect, the present invention provides an isolated ligand that selectively binds to the SARS coronavirus group-specific polypeptide of the first aspect of the invention. For example, the isolated ligand may be an antibody or fragment thereof

In a sixth aspect, the present invention provides a method of identifying a compound that interacts with the SARS coronavirus group-specific polypeptide of the first aspect of the invention, the method comprising the steps of:
(a) contacting the SARS coronavirus group-specific polypeptide with a candidate compound under conditions suitable to permit interaction of the candidate compound to the polypeptide; and
(b) detecting the interaction between said candidate compound and the polypeptide.

The detection of the interaction may comprise adding a labeled substrate and measuring a change in the labeled substrate.

In a seventh aspect, the present invention provides a protein capable of interaction with the SARS coronavirus group-specific polypeptide of the first aspect of the invention.

The protein may be small glutamine-rich tetratricopeptide (SGT) repeat-containing protein. The interaction may comprise degradation of the small glutamine-rich tetratricopeptide (SGT) repeat-containing protein by the SARS coronavirus group-specific polypeptide.

In an eighth aspect, the present invention provides an agent for inhibiting apoptosis by the SARS coronavirus group-specific polypeptide of the first aspect, the agent comprising one or more proteins from the family of Bcl-2 related proteins.

In one embodiment, the one or more proteins may comprise BCl-X_{L}, BAD or combinations thereof.

In a ninth aspect, the present invention provides a method for the treatment and/or prophylaxis of infection by a SARS coronavirus, wherein said method comprises administering a therapeutically effective amount the agent of the eighth aspect of the invention.

In one embodiment, the method of the ninth aspect involves the inhibition of apoptosis associated with the overexpression of the SARS coronavirus group-specific polypeptide of the first aspect of the invention. The inhibition of apoptosis may be achieved by inhibiting the formation of a complex between the SARS coronavirus group-specific polypeptide of the first aspect of the invention and one or more polypeptides. The one or more polypeptides may be SARS coronavirus group-specific polypeptides. The SARS coronavirus group-specific polypeptide may be SARS coronavirus group specific polypeptide 3a.

According to a tenth aspect of the present invention, there is provided a method of identifying a compound that binds to the SARS coronavirus group-specific polypeptide of the first aspect, the method comprising the steps of:
(a) contacting the SARS coronavirus group-specific polypeptide with a candidate compound; and
(b) assaying for the formation of a complex between the candidate compound and the polypeptide.

The assay for the formation of a complex may be selected from the group consisting of a competitive binding assay, a two-hybrid assay and an immunoprecipitation assay.

According to an eleventh aspect of the present invention, there is provided a method of screening for a compound that modulates the activity of the SARS coronavirus group-specific polypeptide of the first aspect, the method comprising the steps of:
(a) contacting the SARS coronavirus group-specific polypeptide with a candidate compound under conditions suitable to enable interaction of the candidate compound to the polypeptide; and
(b) assaying for activity of the polypeptide.

Assaying for activity of the SARS coronavirus group-specific polypeptide may comprise adding a labeled substrate and measuring a change in the labeled substrate. The modulation of activity may be as a result of an inhibition of activity of the SARS coronavirus group-specific polypeptide.

In one embodiment, the method of the sixth, tenth or eleventh aspects, involves identifying compounds which decrease the expression of the polypeptide of the first aspect of the invention. In another embodiment, the compounds identified in accordance with method of the sixth, tenth or eleventh aspects, decreases the expression of the polypeptide of the first aspect of the invention by inhibiting the formation of a complex between the SARS coronavirus group-specific polypeptide of the first aspect and one or more polypeptides, such as SARS coronavirus group-specific polypeptides. For example, interaction with the SARS coronavirus group-specific polypeptide 3a.

According to a twelfth aspect of the present invention, there is provided a method of identifying an agent which is an inhibitor of infection by a SARS coronavirus, the method comprising contacting a cell or cell extract with one or more candidate agents, determining whether there is a decrease in the activity of the SARS coronavirus group-specific polypeptide of the first aspect, and thereby determining whether the agent is an inhibitor of a SARS coronavirus.

According to a thirteenth aspect of the present invention, there is provided a method of identifying an agent suitable for use in the treatment or prevention of SARS coronavirus in a subject, the method comprising:
(a) obtaining a biological sample from the subject,
(b) contacting the sample with a candidate agent,
(c) determining whether there is a decrease in the activity of the polypeptide of the first aspect, and
thereby determining whether the agent is suitable for use in the treatment of SARS coronavirus.

In one embodiment, the method of the twelfth or thirteenth aspects, involves identifying agents which decrease the expression of the polypeptide of the first aspect of the invention. In another embodiment, the compounds identified in accordance with the method of the twelfth or thirteenth aspects, decreases the expression of the polypeptide of the first aspect of the invention by inhibiting the formation of a complex between the SARS coronavirus group-specific polypeptide of the first aspect and one or more polypeptides, such as SARS coronavirus group-specific polypeptides. For example, interaction with the SARS coronavirus group-specific polypeptide 3a.

In a fourteenth aspect, the present invention provides a method for treating or preventing SARS coronavirus in a subject, the method comprising administering to the subject a therapeutically effective amount of the ligand of the fifth aspect or a compound identified by the method of any one of the sixth and tenth to thirteenth aspects of the invention.

In one embodiment, the method of the fourteenth aspect involves the inhibition of apoptosis associated with the overexpression of the polypeptide of the first aspect of the invention. In another embodiment, the inhibition of apoptosis is achieved by inhibiting the formation of a complex between the SARS coronavirus group-specific polypeptide of the first aspect and one or more polypeptides, such as SARS coronavirus group-specific polypeptides. For example, interaction with the SARS coronavirus group-specific polypeptide 3a.

According to a fifteenth aspect of the present invention, there is provided a method of diagnosing SARS coronavirus infection in a subject, the method comprising the steps of:
(a) obtaining a biological sample from the subject; and
(b) assaying for expression of the SARS coronavirus group-specific polypeptide of the first aspect.

Assaying for the expression of the SARS coronavirus group-specific polypeptide may comprise contacting the biological sample with a compound capable of interacting with the polypeptide such that the interaction can be detected. The compound capable of selectively interacting with the polypeptide may be an antibody or fragment thereof to the SARS coronavirus group-specific polypeptide.

According to a sixteenth aspect of the present invention, there is provided a kit comprising the SARS coronavirus group-specific polypeptide of the first aspect of the invention, together with a pharmaceutically acceptable carrier or diluent. Alternatively, or in addition, the kit may contain a ligand or fragment thereof of the fifth aspect, wherein the ligand may be in the form of an antibody or fragment thereof.

The kit may be used for carrying out the methods of the sixth and tenth to fifteenth aspects above, or the methods of seventeenth to eighteenth aspects set out below.

According to a seventeenth aspect of the present invention, there is provided a method for screening a subject for infection by a SARS coronavirus, the method comprising:
(a) obtaining a biological sample from said subject;
(b) contacting said sample with the ligand or fragment thereof of the fifth aspect, and
(c) detecting the presence of the ligand selectively bound to the SARS coronavirus polypeptide of the first aspect.

The sample within which the method of screening is performed may be a plasma, nucleic acid or cell sample.

According to a eighteenth aspect of the present invention, there is provided a method for screening a subject for infection by a SARS coronavirus, the method comprising:
(a) obtaining a biological sample from said subject;
(b) contacting said biological sample from said subject with the nucleic acid encoding the polypeptide as defined in accordance with the first aspect; and
(c) detecting the presence or absence of hybridization between the nucleic acid of said biological sample and the nucleic acid sequence of the first aspect.

In the method of the eighteenth aspect, the nucleic acid sequence corresponds to a region of the nucleic acid sequence which is capable of selectively hybridizing to the nucleic acid encoding the SARS coronavirus group-specific polypeptide of the first aspect. The region may correspond to SEQ ID NO: 1 or a portion thereof. For instance, oligonucleotides as set out in SEQ ID NOS 3 to 16 may be used as primers in a PCR reaction.

The hybridization may occur and be detected through techniques that are standard and routine amongst those skilled in the art, and include southern and northern hybridization, polymerase chain reaction (PCR).

According to a nineteenth aspect of the invention, there is provided a vaccine, wherein said vaccine comprises a polypeptide as defined in accordance with the first aspect of the invention, or a ligand as defined in accordance with the fifth aspect of the invention, together with a pharmaceutically acceptable carrier, adjuvant and/or diluent.

Typically, the vaccine is formulated for administration via an oral, inhalation or parenteral route. More typically, the route of administration is parenteral.

According to a twentieth aspect of the invention, there is provided a method for inducing an immune response in a subject against infection by a SARS coronavirus, comprising administering to said vertebrate an immunologically effective amount of the polypeptide as defined in accordance with the first aspect of the invention, or a ligand as defined in accordance with the fifth aspect of the invention, or a vaccine as defined in accordance with the nineteenth aspect of the invention.

According to a twenty first aspect of the invention, there is provided a method for the treatment and/or prophylaxis of infection by a SARS coronavirus, wherein said method comprises administering a therapeutically effective amount of the vaccine as defined in accordance with the nineteenth aspect of the invention.

In one embodiment, the method of the twenty first aspect involves the inhibition of apoptosis associated with the overexpression of the polypeptide of the first aspect of the invention. In another embodiment, the inhibition of apoptosis is achieved by inhibiting the formation of a complex between the SARS coronavirus group-specific polypeptide of the first aspect and one or more polypeptides, such as other SARS coronavirus group-specific polypeptides. For example, interaction with the SARS coronavirus group-specific polypeptide 3a.

According to a twenty second aspect of the invention, there is provided a method for the treatment and/or prophylaxis of cancer, wherein said method comprises administering a therapeutically effective amount of the SARS coronavirus group-specific polypeptide of the first aspect.

In one embodiment, in accordance with the method of the twenty second aspect, the treatment and/or prophylaxis of infection by a SARS coronavirus comprises promotion of apoptosis associated with the overexpression of the polypeptide of the first aspect of the invention.

According to a twenty third aspect of the invention, there is provided an isolated expressed SARS coronavirus group-specific polypeptide 3b.

The expressed SARS coronavirus group-specific polypeptide 3b may be encoded by the nucleic acid sequence of SEQ ID NO: 17. The expressed SARS coronavirus group-specific polypeptide 3b may have the amino acid sequence as set forth in SEQ ID NO: 18 including one or more conservative amino acid substitutions.

According to a twenty-fourth aspect of the present invention, there is provided a method of identifying an agent which is an inhibitor of infection by a SARS coronavirus, the method comprising contacting a cell or cell extract with one or more candidate agents, determining whether there is a decrease in the activity of the SARS coronavirus group-specific polypeptide of the twenty-third aspect, and thereby determining whether the agent is an inhibitor of a SARS coronavirus.

According to a twenty-fifth aspect of the present invention, there is provided a method of identifying an agent suitable for use in the treatment or prevention of SARS coronavirus in a subject, the method comprising:
(a) obtaining a biological sample from the subject,
(b) contacting the sample with a candidate agent,
(c) determining whether there is a decrease in the activity of the polypeptide of the twenty-third aspect, and thereby determining whether the agent is suitable for use in the treatment of SARS coronavirus.

In a twenty-sixth aspect, the present invention provides a method for treating or preventing SARS coronavirus in a subject, the method comprising administering to the subject a therapeutically effective amount of a compound identified by the method of the twenty-fourth or twenty-fifth aspects of the invention.

In one embodiment, the method of the twenty-sixth aspect involves the inhibition of cell death associated with the overexpression of the polypeptide of the twenty-third aspect of the invention. In another embodiment, the inhibition of cell death is achieved by inhibiting the formation of a complex between the SARS coronavirus group-specific polypeptide of the twenty-third aspect and one or more polypeptides, such as SARS coronavirus group-specific polypeptides.

### DEFINITIONS

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides.

As used herein the term “polypeptide” means a polymer made up of amino acids linked together by peptide bonds.

The term “isolated” means that the material in question has been removed from its host, and associated impurities reduced or eliminated. Essentially, it means an object species is the predominant species present (i.e., on a molar basis it is more abundant than any other individual species in the composition), and preferably a substantially purified fraction is a composition wherein the object species comprises at least about 30 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species.

As used herein “sequence identity” refers to the residues in two sequences that are the same when aligned for maximum correspondence over a specified window of comparison by means of computer programs known in the art such as GAP provided in the GCG program package (Program Manual for the Wisconsin Package, Version 8, August 1996, Genetics Computer Group, 575 Science Drive, Madison, Wis., USA 53711) (Needleman, S. B. and Wunsch, C. D., (1970), Journal of Molecular Biology, 48, 443-453).

The term “conservative amino acid substitution” as used herein refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution.

The term “antibody” means an immunoglobulin molecule able to bind to a specific epitope on an antigen. Antibodies can be comprised of a polyclonal mixture, or may be monoclonal in nature. Further, antibodies can be entire immunoglobulins derived from natural sources, or from recombinant sources. The antibodies of the present invention may exist in a variety of forms, including for example as a whole antibody, or as an antibody fragment, or other immunologically active fragment thereof, such as complementarity determining regions. Similarly, the antibody may exist as an antibody fragment having functional antigen-binding domains, that is, heavy and light chain variable domains. Also, the antibody fragment may exist in a form selected from the group consisting of, but not limited to: Fv, F_{ab}, F(ab)₂, scFv (single chain Fv), dAb (single domain antibody), bi-specific antibodies, diabodies and triabodies.

As used herein the term “treatment”, refers to any and all uses which remedy a disease state or symptoms, prevent the establishment of disease, or otherwise prevent, hinder, retard, or reverse the progression of disease or other undesirable symptoms in any way whatsoever.

As used herein, the term “selectively binds” refers to the ability of antibodies to the SARS coronavirus group-specific polypeptides, such as 7a, to preferentially bind to specified proteins and mimetopes thereof of the present invention. Binding can be measured using a variety of methods standard in the art including enzyme immunoassays (e.g., ELISA), immunoblot assays, etc. An anti-SARS coronavirus group-specific antibody selectively binds to the SARS coronavirus group-specific polypeptide in such a way as to reduce its activity.

As used herein, the term “selectively hybridizes” refers to the ability of nucleic acids, such as probes or primers, of the present invention to preferentially bind to nucleic acids encoding SARS coronavirus group-specific polypeptides, such as 7a. In indicating that a sequence “selectively hybridizes”, the term includes reference to hybridization, under stringent hybridization conditions, to a specific nucleic acid target sequence to a detectably greater degree than a non-target nucleic acid sequence.

As used herein the term “therapeutically effective amount” includes within its meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

In the context of this specification, the term “comprising” means “including principally, but not necessarily solely”. Furthermore, variations of the word “comprising”, such as “comprise” and “comprises”, have correspondingly varied meanings.

### BRIEF DESCRIPTION OF THE DRAWINGS

Preferred aspects of the present invention will now be described, by way of example only, with reference to the accompanying drawings.

FIG. 1. Genome organization of SARS-CoV. ORFs encoding the nonstructural proteins (black boxes), as well as ORFs encoding the structural polypeptides (grey boxes) are indicated (S, Spike; E, Envelope; M, Membrane; N, Nucleocapsid). Also, selected ORFs encoding for putative accessory genes (unshaded boxes) are shown. The ORF7a (ORF7a=X4=8) encoding peptide 7a is represented by the striated box.

FIG. 2. 7a is expressed in SARS CoV-infected Vero E6 cells. (A) Analysis of the 7a putative sequence predicts a signal peptide sequence (underlined) at the N-terminus, the cleavage site of which is indicated with an arrow. A putative membrane-spanning domain (boxed) and an ER retrieval motif (bold-italics) are found at the C-terminus. (B) 20 μg of protein from uninfected Vero E6 cells (lane 1), 20 μg of proteins from SARS-CoV infected Vero E6 cells harvested at about 25% CPE (lane 2), and 50-75% CPE (lane 3); 15 μg total protein from Vero E6 transfected with pXJU122 plasmid (lane 4), 20 μg total lysate from an IBV infected Vero E6 culture (lane 5) was probed with mouse anti-7a antiserum. (C) Uninfected and virus-infected Vero E6 cells at 25% CPE were fixed and stained with mouse anti-7a antibody.

FIG. 3. Processing of 7a expressed in Vero E6 cells. (A) Untagged 7a was in vitro translated and visualized by autoradiography. A single band of ˜17.5 kD was detected. (B) Untagged 7a-L (lane 1), mat7a (lane 2) and 7a (lane 3) proteins were expressed in Vero E6 cells. Total proteins were extracted using RIPA buffer and 20 μg used for 15%-SDS PAGE. Western blots of these proteins were probed with mouse anti-7a antibodies. (C) Vero E6 cells were transfected with pXJU122 for pulse-chase analysis. At 6 h posttranfection cells were starved for 30 min in cysteine- and methionine deficient medium and subsequently labeled for 30 min with [³⁵S]-labeled amino acids. Cells were either lysed directly (0 min) or chased for 30, 60, 90 or 120 min. Cell lysates were immunoprecipitated with mouse anti-7a antibodies, separated on a 15% SDS-PAGE gel followed by autoradiography. (D) Quantification of the pulse-chase experiment. The amount of immature (▴) and mature (o) [³⁵S]-7a was determined with a densitometer and expressed as a percentage of the total labeled protein at each time point (results are means±S.E.M., n=2).

FIG. 4. The 7aK>E protein is rapidly processed in transfected cells. (A) Untagged 7a and 7aK>E proteins were expressed in transfected Vero E6 cells. Sixteen hours posttransfection total proteins were extracted using RIPA buffer and 20 μg separated on 15%-SDS PAGE gel. Western blots of these proteins were probed with mouse anti-7a antibodies. (B) Vero E6 cells were transfected with pXJU122K>E for pulse-chase analysis. At 6 h posttranfection cells were starved for 30 min in cysteine- and methionine-deficient medium and subsequently labeled for 10 min with [³⁵S]-labeled amino acids. Cells were either lysed directly (0 min) or chased for 10, 20, 30 or 60 mins. Cell lysates were immunoprecipitated with mouse anti-7a antibodies followed by separation on a 15% SDS-PAGE gel and autoradiography.

FIG. 5. Intracellular localization of expressed 7a and retrieval signal mutant. Vero E6 cells were transfected with pXJU122 or pXJU122K>E. At 16 h posttransfection, cells were fixed with methanol and labeled with mouse anti-7a antibody (left panels) and either antibodies to the ER marker GRP94 (rat anti-GRP94), the intermediate compartment marker Sec31 (rabbit anti-Sec31) or Golgi marker GS28 (rabbit anti-GS28) (middle panels). When anti-7a was used with anti-GS28 or anti-Sec31, FITC-conjugated goat anti-mouse and Rh-conjugated anti-rabbit antibodies (Santa Cruz Biochemicals, USA) were used as secondary antibodies. For double-labeling with anti-7a and anti-GRP94, FITC-conjugated anti-rat and Rh-conjugated anti-mouse (Santa Cruz Biochemicals, USA) antibodies were used as secondary antibodies. Merged images showed co-localization of 7a and K>E proteins with the markers proteins (right panels)

FIG. 6. Expression of the viral proteins in 293 T cells and the effects on apoptosis. (A) CaspACE fluorometric assay system from Promega Corporation (Madison, Wis., USA) was used to measure the activation of caspase-3 protease activity, which is a hallmark of apoptosis, in cells that were transfected with a positive control (HA-BAX, column 1), a negative control (HA-GST, column 2), and the different SARS-CoV proteins (columns 3 to 7). All experiments were performed in duplicates and the average values with standard deviations are plotted. For cell viability assays, experiments were performed in triplicates and the average percentages (±standard deviations) of live cells, compared to HA-GST which is normalized to 100%, are shown in parentheses above each column. (B) Western blot analysis to determine the cleavage of endogenous full-length PARP, which is a substrate of activated caspase-3, from 116 kDa to 83 kDa (upper panel). Expression levels of the HA-tagged proteins were determined with anti-HA antibody (middle panel) and the amounts of total cell lysates loaded were verified by measuring the level of endogenous actin (lower panel). (C) CaspACE fluorometric assay system from Promega Corporation (Madison, Wis., USA) was used to measure the activation of caspase 3 in cells that were transfected 7a-HA, 7a and HA-BAX in the presence of pan-caspase inhibitor, zVAD-fink (columns 2 to 4) or an irrelevant peptide, zFA-fmk (columns 5 to 7). (D) Western blot analysis were performed to determine the cleavage of endogenous PARP (upper panel), expression levels of HA-GST, 7a-HA, 7a and HA-BAX (anti-7a or anti-HA, middle panels) and endogenous actin as a loading control (anti-actin, lower panel). As the anti-7a antibody was obtained using a GST-fusion protein, it recognizes both 7a and GST proteins.

FIG. 7. Induction of apoptosis by 7a in cell-lines derived from different organs. The cell-lines used were HeLa, cervical carcinoma (lanes 1 to 3); HepG2, liver carcinoma (lanes 4 to 6); A549, lung carcinoma (lanes 7 to 9); COS7 and Vero E6, kidney (lanes 10 to 12 and lanes 13 to 15, respectively). All cell-lines were purchased from the American Type Culture Collection (Manassas, Va., USA). CaspACE fluorometric assay system from Promega Corporation (Madison, Wis., USA) was used to measure the activation of caspase-3 protease in different cell-lines that were transfected HA-GST, 7a-HA and HA-BAX (1^{st} panel). Western blot analysis were performed to determine the cleavage of endogenous PARP (2^{nd} panel), expression levels of HA-GST, 7a-HA and HA-BAX (anti-HA, 3^{rd} panel) and endogenous actin as a loading control (anti-actin, 4^{th} panel).

FIG. 8. Induction of apoptosis by overexpression of wild-type 7a and 7a mutants (7a-L and mat7a) in A549 and Vero E6 cells and by SARS-CoV infection of Vero E6 cells. (A) CaspACE fluorometric assay system from Promega Corporation (Madison, Wis., USA) was used to measure the activation of caspase-3 protease in different cell-lines that were transfected with HA-GST (negative control, lanes 1 and 5), wild-type 7a (lanes 2 and 6), mutant 7a-L (lanes 3 and 7), and mutant mat7a (lanes 4 and 8) (1^{st} panel). 7a-L contains mutations at the signal peptide cleavage site and is cleaved less efficiently than wild-type and mat7a does not contain the signal peptide. Western blot analyses were performed to determine the expression levels of GST and 7a proteins (anti-7a, 2^{nd} panel) and endogenous actin as a loading control (anti-actin, 3^{rd} panel). (B) Caspase-3 protease activities in Vero E6 cells transfected with 1.0 μg (lane 1), or 2.0 μg (lane 2) of 7a plasmid or mocked infected cells (lane 3), or SARS-CoV infected cells (lane 4), were determined (1^{st} panel). Western blot analyses were performed to determine the expression levels of 7a (anti-7a, 2^{nd} panel), SARS-CoV N and endogenous tubulin as a loading control (monoclonal anti-tubulin (Sigma), 4^{th} panel) in 20 μg of cell lysates.

FIG. 9: Co-immunoprecipitation experiments (A) 293T cells were transiently transfected with pXJ40myc-GST or pXJ40myc-7a and the various HA-tagged constructs. After ˜16 to 24 h, the cells were harvested and used in co-immunoprecipitation experiments as previously described. Briefly, the lysate was incubated with an anti-myc polyclonal antibody (Santa Cruz Biotechnology) for 2 h at 4° C., followed by adsorption onto 10 μl of protein A-sepharose beads (Roche Molecular Biochemicals). Then the beads were washed 3 times with cold reaction buffer, and subjected to Western blot analysis. In the figure, lanes 1, 3, 5, 7, 9 represent co-transfection of myc-GST with HA-N, M-HA, E-HA, 7a-HA and 3a-HA respectively. And lanes 2, 4, 6, 8, 10 represent co-transfection of 7a-myc with HA-N, M-HA, E-HA, 7a-HA and 3a-HA respectively. (B) Interaction of 7a with other SARS-CoV proteins. Co-immunoprecipitation experiments were performed to determine if 7a could interact with the SARS-CoV structural proteins, M, E and N, as well as with 3a and with itself. As shown in the figure (upper panel, immunoprecipitation (IP)), myc-7a specifically co-immunoprecipitated E-HA (lane 6) and M-HA (lane 4) and neither of these showed any unspecific binding to myc-GST (lanes 5 and 3). HA-N did not interact with myc-7a or myc-GST (lanes 1 and 2). 7a also interacts strongly and specifically with 3a (lanes 9 and 10), another unique SARS-CoV protein that is expressed in infected cells. As shown in lanes 7 and 8, 7a-myc can specifically immunoprecipitate 7a-HA, suggesting that 7a can also form dimers or oligomers. Panels 2 and 3 show the expression levels of the different proteins by Western blot analysis (WB).

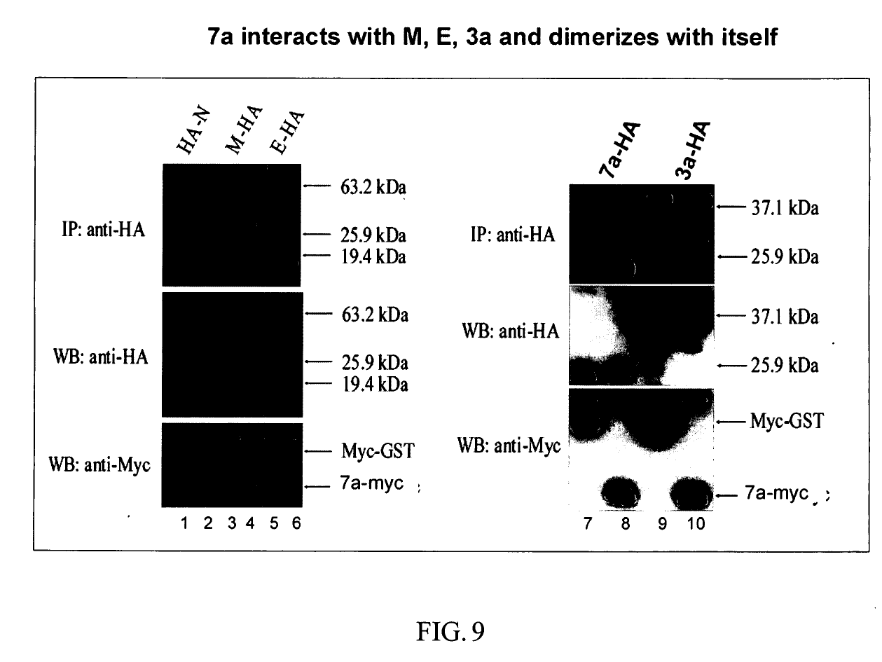

FIG. 10: Overexpression of Bcl-X_{L} prevents the induction of apoptosis by SARS-CoV 7a (A) The expression of Bcl-X_{L} in 293T cells (293T-Vec, clone 1A, lane 1) and 293T cells stably expressing BC1-X_{L} (293T-Bcl-X_{L}, clone 10A, lane 2) was determined by Western blot analysis using an anti-Bcl-X_{L} antibody (1ₛₜ panel). An unknown cellular protein cross-reacting with the anti-Bcl-X_{L} antibody is marked with an asterisk. Equal amounts of cells were used in each lane as verified by the level of endogenous actin (2^{nd} panel). (B) CaspACE fluorometric assay system from Promega Corporation (Madison, Wis., USA) was used to measure the activation of caspase-3 protease activity, which is a hallmark of apoptosis, in cells that were transfected with a negative control (HA-GST, columns 1 and 4), a positive control (HA-BAX, columns 2 and 5) and with SARS-CoV 7a protein (columns 3 and 6). Columns 1 to 3 shows the results obtained using 293TVec cells and columns 4 to 6 shows the results obtained using 293T-Bcl-X_{L} cells. All experiments were performed in duplicates and the average values with standard deviations are plotted.

FIG. 11: SARS-CoV 7a interacts specifically with Bcl-X_{L} and BAD. Cell lysates containing myc-GST (negative control) or different myc-tagged Bcl-2 family proteins (myc-Bcl-X_{L}, myc-BID, myc-BAD, myc-BAX, myc-BAK) with SARS-CoV 7a protein were immunoprecipitated with anti-SARS-CoV 7a antibody and protein A beads. The amounts of SARS-CoV 7a protein that co-immunoprecipitated (IP) with the myc-tagged proteins were determined with an anti-SARS-CoV 7a antibody (1ₛₜ panel). Expression levels of the SARS-CoV 7a protein were determined by subjecting aliquots of the lysates before immunoprecipation to Western blot analysis with an anti-SARS-CoV 7a antibody (2_{nd} panel). The first blot containing the immuno-complexes (i.e. upper panel) was stripped and re-probed with an anti-myc monoclonal antibody to determine the amount of myc-tagged proteins immunoprecipitated (3_{rd} panel).

FIG. 12: Binding of SGT to 7a-HA in Vero E6 cells. (A) Rabbit anti-SGT specifically detected endogenous mSGT from Vero E6 cells, as well as transfected flag-hSGT. (B) Co-immunoprecipitation of flag-SGT and mSGT with 7A-HA. Lysates from Vero E6 cells transfected with flag-SGT and/or 7a-HA were extracted and immunoprecipitated (IP) with rabbit anti-flag (lane 1), rabbit anti-SGT (lane 2 and 4) or rabbit anti-myc antibody (lane 3) conjugated to Protein-A beads, respectively. IP samples (upper panel), together with direct lysates (WB—lower panel) were analyzed by 15% SDS-PAGE and Western blotting with rabbit anti-HA. (C) Vero E6 cells were transfected with 7a and flag-hSGT. At 16 hours post-transfection, cells were lysed. Western blots of direct lysates from the transfected cells were incubated with mouse anti-flag antibody; actin was included as a loading control.

FIG. 13: Comparison of the amino acid sequence of monkey SGT with a human homologue. Conceptual amino acid sequence of mSGT was compared to the amino acid sequence of hSGT (NCBI accession number NP_{—}003012) using CLUSTAL X. Results were visualized with GENEDOC software. Identical residues are represented with black shading.

FIG. 14: Co-localization of flag-SGT and 7a-HA in Vero E6 cells. Vero E 6 cells were grown on cover-slips and transfected with pXJ3′-7a-HA and pXJ40flag-SGT. At 16 hrs post-transfection, the cells were fixed and co-stained with both rabbit anti-flag and mouse anti-HA. Following washes, the cells were treated with FITC-conjugated anti-rabbit secondary antibody and rhodamine-conjugated (Rh) anti-mouse antibody. Primary antibodies were used at dilutions of 1:200 and secondary antibodies at 1:100. Cover-slips were mounted on glass-slides and viewed by immunofluorescence. Panels on the left represent cells stained with FITC, those in the centre panels with Rh and composite photos are shown in the right panels.

FIG. 15: hSGT interacts with the C-terminal region of 7a-HA. (A) Schematic representation of 7a-HA deletion mutants used in this study. Full length 7a-HA (aa 1-122), 7aΔC-HA (aa 1-61) and 7aΔN-HA (aa 62-122) are shown. All proteins were HA-tagged at the C-terminus. (B) HA-tagged full length or mutant 7a-HA was co-transfected into Vero-E6 cells with flag-hSGT, respectively. Total proteins were extracted and co-immunoprecipitated with mouse anti-flag beads. The name of each clone is shown on the left and results of the co-IP are shown on the right. (striated box=signal peptide; lined box=transmembrane domain; black box=ER-Golgi retrieval signal motif).

FIG. 16: TPR 2 is essential for the interaction with SARS-CoV 7a-HA. (A) Full length flag-hSGT (aa 1-313) and deletion mutants flag-SGTΔN-1 (aa 125-313), flag-SGTΔC (aa 1- 192), flag-SGTΔC-3 (aa 1-158), flag-SGTΔC3-2 (aa 1-124) and flag-SGTΔC3-1 (aa 1-91) are shown. All hSGT proteins were flag-tagged at the N-terminus. The locations of the TPR motifs 1, 2 and 3 are represented by shaded boxes and co-IP results are shown on the right. The name of each clone is shown on the left and results of the co-IP are shown on the right. (B) Flag-tagged full length or mutant hSGT were co-transfected into Vero-E6 cells with 7a- HA, respectively. Total proteins were extracted and co-immunoprecipitated with mouse antiflag beads. IP complexes were Western Blotted and probed with rabbit anti-flag or rabbit anti-HA antibodies.

FIG. 17: 7a interaction with SAR-CoV structural proteins. Co-immunoprecipitation analysis between 7a-myc and SARS-CoV HA-epitope tagged structural proteins N, M and E. GST-HA was included as negative control.

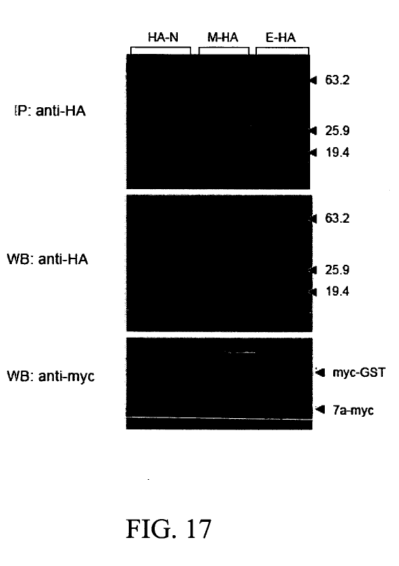

FIG. 18. Induction of cell-death/apoptosis by SARS-CoV 3b protein. (A) CaspACE fluorometric assay system from Promega Corporation (Madison, Wis., USA) was used to measure activation of caspase-3 protease activity, which is a hallmark of apoptosis, in untransfected cells (column 1) or cells that were transfected with a negative control (HA-GST, column 2), or a positive control (HA-BAX, column 3), or SARS-CoV 7a proteins (columns 4 to 5) or SARS-CoV 3b proteins (columns 7 to 9). All experiments were performed in duplicates and the average values with standard deviations were plotted. (B) Western blot analysis was carried out to determine the cleavage of endogenous full-length PARP, which is a substrate of activated caspase-3, from 116 kDa to 83 kDa (1st panel). The antibody used is specific for the cleaved form of PARP (i.e. 83 kDa) that is produced during apoptosis. The amounts of total cell lysates loaded were verified by measuring the level of endogenous actin (2nd panel). Expression levels of different proteins were determined by probing with the respective antibodies (3rd, 4th and 5th panels).

FIG. 19: (A) shows the level of caspase 3 activity in cells transiently transfected with cDNA for expressing HA-tagged SARS CoV 3b protein (column 1), Bax protein (column 2) or empty vector (column 3). MI represents untransfected cells (column 4). Both SARS-CoV 3b and Bax induce apoptosis as shown by the higher levels of caspase 3 activities compared to control cells (columns 3 and 4), but the level induced by 3b is significantly lower than Bax. (B) shows the level of cell death, as measured by the leakage of LDH into culture medium, for cells transfected in a similar manner as in (A). Both SARS-CoV 3b and Bax induce similar level of cell-death. Our results showed that SARS-CoV 3b can induce cell-death by both caspase-3 dependent and independent pathways.

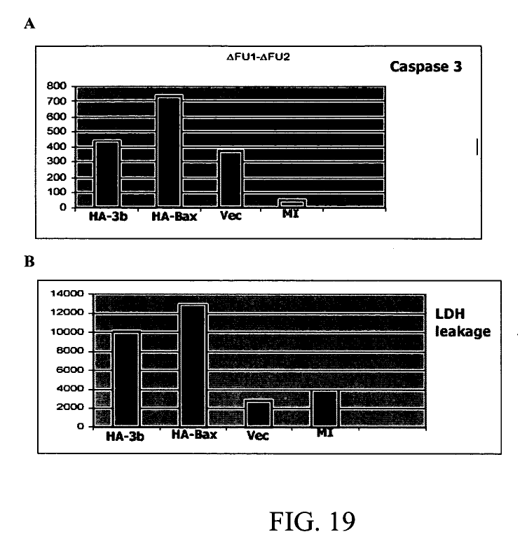

### DETAILED DESCRIPTION OF THE INVENTION

Nucleic Acid Sequence

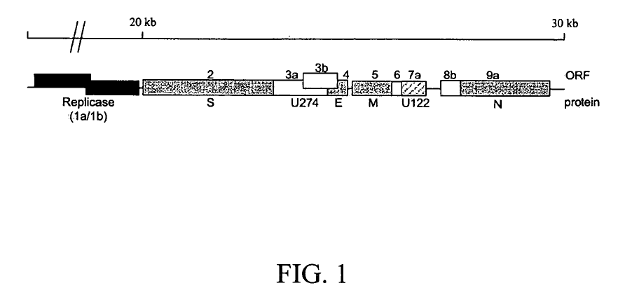

There is described herein the characterization of the SARS-CoV group-specific gene product encoded by ORF7a (also known as ORFX4 or ORF8) (FIG. 1) which is referred to as 7a (involving a unique protein of 122 amino acids). To assist in understanding the role that 7a plays in the infectivity of SARS-CoV, characterization of the gene is set out below.

Typically, the nucleic acid molecule encoding the SARS coronavirus group-specific polypeptide of the first aspect also includes within its scope a variant or fragment of the nucleic acid sequence, wherein said variant or fragment encodes a polypeptide having a biological activity which is functionally the same as the polypeptide (or fragment thereof) encoded by the nucleic acid molecule of the invention, in particular the nucleic acid sequence defined in SEQ ID NO:1 wherein said variant can be located and isolated using standard techniques in molecular biology, without undue trial and experimentation.

The degree of homology between two nucleic acid sequences may be determined by means of computer programs known in the art such as GAP provided in the GCG program package (Program Manual for the Wisconsin Package, Version 8, August 1996, Genetics Computer Group, 575 Science Drive, Madison, Wis., USA 53711) (Needleman, S. B. and Wunsch, C. D., (1970), Journal of Molecular Biology, 48, 443-453). Using GAP with the following settings for DNA sequence comparison: GAP creation penalty of 5.0 and GAP extension penalty of 0.3.

Nucleic acid molecules may be aligned to each other using the Pileup alignment software, available as part of the GCG program package, using, for instance, the default settings of gap creation penalty of 5 and gap width penalty of 0.3.

The nucleic acid molecule may also include within its scope a variant capable of hybridizing to the nucleic acid molecules of the invention, in particular the nucleic acid sequences defined in SEQ ID NO:1 under conditions of low stringency, more preferably, medium stringency and still more preferably, high stringency. Low stringency hybridization conditions may correspond to hybridization performed at 50° C. in 2×SSC.

Suitable experimental conditions for determining whether a given nucleic acid molecule hybridizes to a specified nucleic acid may involve presoaking of a filter containing a relevant sample of the nucleic acid to be examined in 5×SSC for 10 min, and prehybridization of the filter in a solution of 5×SSC, 5×Denhardt's solution, 0.5% SDS and 100 μg/ml of denatured sonicated salmon sperm DNA, followed by hybridization in the same solution containing a concentration of 10 ng/ml of a ³²P-dCTP-labeled probe for 12 hours at approximately 45° C., in accordance with the hybridization methods as described in Sambrook et al. (1989; Molecular Cloning, A Laboratory Manual, 2nd edition, Cold Spring Harbour, N.Y.).

The filter is then washed twice for 30 minutes in 2×SSC, 0.5% SDS at least 55° C. (low stringency), at least 60° C. (medium stringency), at least 65° C. (medium/high stringency), at least 70° C. (high stringency), or at least 75° C. (very high stringency). Hybridisation may be detected by exposure of the filter to an x-ray film.

Further, there are numerous conditions and factors, well known to those skilled in the art, which may be employed to alter the stringency of hybridization. For instance, the length and nature (DNA, RNA, base composition) of the nucleic acid to be hybridized to a specified nucleic acid; concentration of salts and other components, such as the presence or absence of formamide, dextran sulfate, polyethylene glycol etc; and altering the temperature of the hybridization and/or washing steps.

Further, it is also possible to theoretically predict whether or not two given nucleic acid sequences will hybridize under certain specified conditions. Accordingly, as an alternative to the empirical method described above, the determination as to whether a variant nucleic acid sequence will hybridize to the nucleic acid molecule defined in accordance with the first aspect, or more specifically, the nucleic acid of SEQ ID NO:1, can be based on a theoretical calculation of the Tₘ (melting temperature) at which two heterologous nucleic acid sequences with known sequences will hybridize under specified conditions, such as salt concentration and temperature.

In determining the melting temperature for heterologous nucleic acid sequences (Tₘ₍ₕₑₜₑᵣₒ₎) it is necessary first to determine the melting temperature (Tₘ₍ₕₒₘₒ₎) for homologous nucleic acid sequence. The melting temperature (Tₘ₍ₕₒₘₒ₎) between two fully complementary nucleic acid strands (homoduplex formation) may be determined in accordance with the following formula, as outlined in Current Protocols in Molecular Biology, John Wiley and Sons, 1995, as:
Tₘ₍ₕₒₘₒ₎=81.5° C.+16.6 (log M)+0.41 (% GC)−0.61 (% form)−500/L
M=denotes the molarity of monovalent cations,
% GC=% guanine (G) and cytosine (C) of total number of bases in the sequence,
% form=% formamide in the hybridization buffer, and
L=the length of the nucleic acid sequence.

Tₘ determined by the above formula is the Tₘ of a homoduplex formation (Tₘ₍ₕₒₘₒ₎) between two fully complementary nucleic acid sequences. In order to adapt the Tₘ value to that of two heterologous nucleic acid sequences, it is assumed that a 1% difference in nucleotide sequence between two heterologous sequences equals a 1° C. decrease in Tₘ. Therefore, the Tₘ₍ₕₑₜₑᵣₒ₎ for the heteroduplex formation is obtained through subtracting the homology % difference between the analogous sequence in question and the nucleotide probe described above from the Tₘ₍ₕₒₘₒ₎.

Typically the nucleic acid molecule defined in SEQ ID NO:1 also includes within its scope a nucleic acid molecule which is an oligonucleotide fragment thereof. Typically, the oligonucleotide fragment is between about 8 to about 375 nucleotides in length. More typically, the oligonucleotide fragment is between about 8 to about 250 nucleotides in length. Even more typically still, the oligonucleotide fragment is between about 8 to about 125 nucleotides in length. Yet still more typically, the oligonucleotide fragment is about 150 nucleotides in length.

Polypeptide

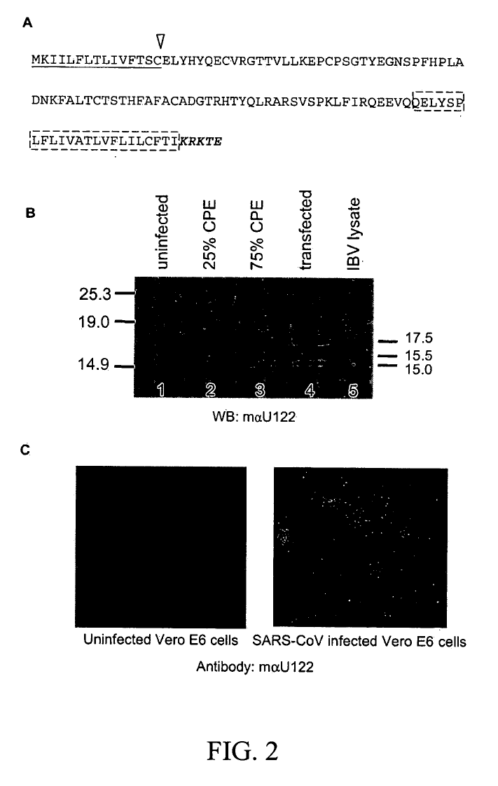

In considering the SARS coronavirus group specific polypeptide 7a, sequence analysis predicted a 122 amino acid polypeptide, with a putative signal peptide sequence, C terminus transmembrane domain, and short cytoplasmic tail containing the endoplasmic reticulum (ER) retrieval motif KRKTE (FIG. 2A). Using Western blot and immunofluorescence, it is apparent that 7a was expressed in SARS-CoV-infected cells. The initial characterization of the localization and processing of 7a is presented herein, and additionally, mutational analysis was used to characterize the putative signal peptide and ER retrieval sequences, and this is set out below in more detail in the examples.

The SARS coronavirus group-specific polypeptide of the of the first aspect of the invention also includes within its scope a fragment or variant of the polypeptide sequence, wherein said variant or fragment reflects a polypeptide having a biological activity which is functionally the same as the polypeptide of the first aspect. For instance, variant or fragment reflects a polypeptide which is sufficiently immunogenic to produce an immune response against the polypeptide of the first aspect. The term “fragment” in the context of a polypeptide refers to a polypeptide sequence that is a constituent of the full-length SARS coronavirus group-specific polypeptide. The term “variant” refers to a substantially similar sequence to that of SARS coronavirus group-specific polypeptide. Generally, variant polypeptides may share at least 60%, 65%, 70%, 75%, 80%, 85%, 90%, 95%, 96%, 97%, 98% or 99% sequence identity. Further, a variant polypeptide may include an analogue, wherein the term “analogue” means a polypeptide which is a derivative of a SARS coronavirus group-specific polypeptide, which derivative comprises addition, deletion or substitution of one or more amino acids such that the polypeptide retains substantially the same function as native SARS coronavirus group-specific polypeptide. The term “conservative amino acid substitution” refers to a substitution or a placement of one amino acid for another with similar properties within a polypeptide chain

In vitro detection of the polypeptides or variants or fragments thereof of the present invention may be achieved using a variety of techniques including ELISA (enzyme linked immunosorbent assay), Western blotting, immunoprecipitation and immunofluorescence. Such techniques are commonly used by those of skill in the art. Similarly, suitable techniques of the in vivo detection of the polypeptide, or fragments or analogues thereof, including immunohistochemistry using a labeled anti-SARS coronavirus group-specific polypeptide, will be readily understood by persons skilled in the art.

In accordance with the present invention, fusion proteins may also be engineered to improve characteristics of the SARS coronavirus group-specific polypeptide. For example, a region of additional amino acids, particularly charged amino acids, may be added to the N-terminus of the SARS coronavirus group-specific polypeptide to improve stability during purification from a host cell. Alternatively, peptide moieties may be added to the polypeptide to facilitate purification. Such regions may be removed prior to final preparation of the polypeptide. The addition of peptide moieties to facilitate handling of polypeptides are routine techniques well known to those of skill in the art.

Typically the polypeptide defined in accordance with the first aspect wherein such a polypeptide includes within its scope that set out in SEQ ID NO: 2, also includes within its scope a peptide fragment thereof. The peptide fragment may be between about 5 to about 150 amino acids in length. Alternatively, the peptide fragment may be between about 8 to about 100 amino acids in length. Alternatively, the peptide fragment may be between about 8 to about 50 amino acids in length. Alternatively, the peptide fragment may be between about 8 to about 35 amino acids in length. Alternatively, the peptide fragment may be between about 8 to about 25 amino acids in length

Uses of the SARS Coronavirus Group-Specific Polypeptides

Antibodies

In preparing antibodies for use in the invention, a general method, such as that set out in Harlow, E. and D. Lane. 1988 Antibodies: a laboratory manual, Cold Spring Harbor Laboratory, New York, U.S.A. (the disclosure of which is incorporated herein by reference) may be used. The antibody may be a polyclonal or monoclonal antibody preparation, identifiable by a person skilled in the art upon reading of the present disclosure. Monoclonal antibodies, which are homogenous populations of antibodies to a particular antigen, may be obtained by any technique that provides for the production of antibody molecules by continuous cell line culture. These techniques are well known and routinely used in academic and industrial settings. Producing monoclonal Antibodies requires immunizing an animal, such as a mouse or rabbit or any other animal; obtaining the immune cells from the animals; and fusing the cells with an immortal cell such as a cancer cell, a stem cell or any other continuous cell. This fused cell then becomes an immortal cell line, which means that it will grow and divide indefinitely like a tumor. The tumor of the immortal fused cells is called a hybridoma, and these cells produce monoclonal Antibodies that are then isolated from the hybridomas. Some techniques include but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl Acad. Sci. USA 80:2026-2030), and the EBV-hybridoma technique (Cole et al., 1985, Monoclonal Antibodies And Cancer Therapy, Alen R. Liss, Inc., pp77-96).]

For example, the purified SARS coronavirus group-specific protein may be used to generate rabbit polyclonal antiserum using methods known in the art, such as, a commercial production service. In addition to whole SARS coronavirus group-specific polypeptide, unique synthetic peptides, such as peptides whose amino acid sequence overlaps all or part of the unique SARS coronavirus group-specific polypeptide may be used as immunogens to generate rabbit polyclonal antiserum. The specificity of the resulting antibodies will be determined by immunoblotting against purified SARS coronavirus group-specific polypeptide. Based upon these results, monoclonal antibodies can be generated against whole or a part of the SARS coronavirus group-specific polypeptide.

The present invention provides antibodies or fragments thereof that selectively bind to the SARS coronavirus group-specific polypeptide of the present invention, as well as variants or fragments thereof. Suitable antibodies include, but are not limited to polyclonal, monoclonal, chimeric, humanised, single chain, Fab fragments, and an Fab expression library. Antibodies of the present invention may act as agonists or antagonists of the SARS coronavirus group-specific polypeptide. Preferably antibodies are prepared from discrete regions or fragments of the SARS coronavirus group-specific polypeptide, in particular those involved in conferring apoptosis activity.

Methods for the generation of suitable antibodies will be readily appreciated by those skilled in the art. For example, an anti-SARS coronavirus group-specific polypeptide monoclonal antibody, typically containing Fab portions, may be prepared using the hybridoma technology described in Antibodies-A Laboratory Manual, Harlow and Lane, eds., Cold Spring Harbor Laboratory, N.Y. (1988).

In essence, in the preparation of monoclonal antibodies directed toward SARS coronavirus group-specific polypeptides, such as SARS coronavirus 7a or 3a proteins, any technique that provides for the production of antibody molecules by continuous cell lines in culture may be used. These again include the hybridoma technique originally developed by Kohler et al., Nature, 256:495-497 (1975), as well as the trioma technique, the human B-cell hybridoma technique [Kozbor et al., Immunology Today, 4:72 (1983)], and the EBV-hybridoma technique to produce human monoclonal antibodies [Cole et al., in Monoclonal Antibodies and Cancer Therapy, pp. 77-96, Alan R. Liss, Inc., (1985)]. Immortal, antibody-producing cell lines can be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies and T-cell Hybridomas” (1981); Kennett et al., “Monoclonal Antibodies” (1980).

In summary, a means of producing a hybridoma from which the monoclonal antibody is produced, a myeloma or other self-perpetuating cell line is fused with lymphocytes obtained from the spleen of a mammal hyperimmunised with a recognition factor-binding portion thereof, or recognition factor, or an origin-specific DNA-binding portion thereof. Hybridomas producing a monoclonal antibody useful in practicing this invention are identified by their ability to immunoreact with the present recognition factor and their ability to inhibit specified transcriptional activity in target cells.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Similarly, there are various procedures known in the art which may be used for the production of polyclonal antibodies to the SARS coronavirus polypeptides. For the production of a SARS coronavirus polyclonal antibody, various host animals can be immunized by injection with the SARS coronavirus polypeptides, such as 7a protein or a fragment or variant thereof, including but not limited to rabbits, mice, rats, sheep, goats, etc.

Further, the SARS coronavirus polypeptides, such as 7a protein or fragment or variant thereof can be conjugated to an immunogenic carrier, e.g., bovine serum albumin (BSA) or keyhole limpet hemocyanin (KLH). Also, various adjuvants may be used to increase the immunological response, including but not limited to Freund's (complete and incomplete), mineral gels such as aluminium hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

Screening for the desired SARS coronavirus antibody can also be accomplished by a variety of techniques known in the art. Assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, and immunoelectrophoresis assays, and the like (see, for example, Ausubel et al., eds, 1994, Current Protocols in Molecular Biology, Vol. 1, John Wiley & Sons, Inc., New York). Antibody binding may be detected by virtue of a detectable label on the primary anti latent phase cytomegalovirus polypeptide antibody. Alternatively, the anti-SARS coronavirus polypeptide antibody may be detected by virtue of its binding with a secondary antibody or reagent which is appropriately labelled. A variety of methods are known in the art for detecting binding in an immunoassay and are within the scope of the present invention.

Antibodies of the present invention can be used in diagnostic methods and kits that are well known to those of ordinary skill in the art to diagnose or determine predisposition to a SARS infection in a subject.

The antibody or fragment thereof raised against the SARS coronavirus polypeptide has binding affinity for the polypeptide. Preferably, the antibody or fragment thereof has binding affinity or avidity greater than about 10⁵ M⁻¹, more preferably greater than about 10⁶ M⁻¹, more preferably still greater than about 10⁷ M⁻¹ and most preferably greater than about 10⁸ M⁻¹.

In terms of obtaining a suitable amount of an antibody according to the present invention, one may manufacture the antibody(s) using batch fermentation with serum free medium. After fermentation the antibody may be purified via a multistep procedure incorporating chromatography and viral inactivation/removal steps. For instance, the antibody may be first separated by Protein A affinity chromatography and then treated with solvent/detergent to inactivate any lipid enveloped viruses. Further purification, typically by anion and cation exchange chromatography may be used to remove residual proteins, solvents/detergents and nucleic acids. The purified antibody may be further purified and formulated into 0.9% saline using gel filtration columns. The formulated bulk preparation may then be sterilised and viral filtered and dispensed.

In a related aspect, the invention may feature a monoclonal antibody, or an Fab, (Fab)₂, scFv (single chain Fv), dAb (single domain antibody), bi-specific antibodies, diabodies and triabodies, or other immunologically active fragment thereof (eg., a CDR-region). Such fragments are useful as immunosuppressive agents. Alternatively, the antibody of the invention may have attached to it an effector or reporter molecule. For instance, an antibody or fragment thereof of the invention may have a macrocycle, for chelating a heavy metal atom, or a toxin, such as ricin, attached to it by a covalent bridging structure. In addition, the Fc fragment or CH₃ domain of a complete antibody molecule may be replaced or conjugated by an enzyme or toxin molecule, such as chelates, toxins, drugs or prodrugs, and a part of the immunoglobulin chain may be bonded with a polypeptide effector or reporter molecule, such as biotin, fluorochromes, phosphatases and peroxidases. Bispecific antibodies may also be produced in accordance with standard procedures well known to those skilled in the art.

The present invention further contemplates genetically modifying the antibody variable and/or constant regions to include effectively homologous variable and constant region amino acid sequences. Generally, changes in the variable region will be made to improve or otherwise modify antigen binding properties of the antibody or fragment thereof. Changes in the constant region will, in general, be made in order to improve or otherwise modify biological properties, such as complement fixation, interaction with membranes, and other effector functions.

In the present context, effectively homologous refers to the concept that differences in the primary structure of the variable region of the antibody or fragment thereof may not alter the binding characteristics of the antibody or fragment thereof. Changes of amino acids are permissable in effectively homologous sequences so long as the resultant antibody or fragment thereof retains its desired property.

Amino acid changes in the polypeptide or the antibody or fragment thereof may be effected by techniques well known to persons skilled in the relevant art. For example, amino acid changes may be effected by nucleotide replacement techniques which include the addition, deletion or substitution of nucleotides, under the proviso that the proper reading frame is maintained. Exemplary techniques include random mutagenesis, site-directed mutagenesis, oligonucleotide-mediated or polynucleotide-mediated mutagenesis, deletion of selected region(s) through the use of existing or engineered restriction enzyme sites, and the polymerase chain reaction.

Modulator and Inhibitor Compounds

In addition to specific anti-SARS coronavirus polypeptides, the polypeptides of the present invention are particularly useful for the screening and identification of compounds and agents that interact with the SARS coronavirus. In particular, desirable compounds include those that decrease the activity of the SARS coronavirus, for example, via inhibiting the overexpression of 7a and apoptosis associated therewith. Such compounds may modulate by inhibiting or preventing SARS viral pathogenesis. Suitable compounds may exert their effect by virtue of either a direct (for example binding) or indirect interaction.

Compounds which bind, or otherwise interact with the SARS coronavirus, and specifically compounds which modulate the activity of the SARS coronavirus, may be identified by a variety of suitable methods. Interaction and/or binding may be determined using standard competitive binding assays or two-hybrid assay systems.

For example, the two-hybrid assay is a yeast-based genetic assay system (Fields and Song, 1989) typically used for detecting protein-protein interactions. Briefly, this assay takes advantage of the multi-domain nature of transcriptional activators. For example, the DNA-binding domain of a known transcriptional activator may be fused to the SARS coronavirus polypeptides, such as 7a protein, and the activation domain of the transcriptional activator fused to a candidate protein. Interaction between the candidate protein and the SARS coronavirus polypeptide, will bring the DNA-binding and activation domains of the transcriptional activator into close proximity. Interaction can thus be detected by virtue of transcription of a specific reporter gene activated by the transcriptional activator.

Alternatively, affinity chromatography may be used to identify SARS coronavirus polypeptide binding partners. For example, SARS coronavirus polypeptides, such as 7a, may be immobilised on a support (such as sepharose) and cell lysates passed over the column. Proteins binding to the immobilised SARS coronavirus polypeptide can then be eluted from the column and identified. Initially such proteins may be identified by N-terminal amino acid sequencing for example.

Methods for detecting compounds that modulate the activity of SARS coronavirus polypeptides, such as 7a, homologue activity may involve combining the SARS coronavirus polypeptide with a candidate compound and a suitable labelled substrate and monitoring the effect of the compound on the SARS coronavirus polypeptide by changes in the substrate (may be determined as a function of time). Suitable labelled substrates include those labelled for colourimetric, radiometric, fluorimetric or fluorescent resonance energy transfer (FRET) based methods, for example. Alternatively, compounds that modulate the activity of the SARS coronavirus polypeptide may be identified by comparing the catalytic activity of SARS coronavirus polypeptide in the presence of a candidate compound with the catalytic activity of the SARS coronavirus polypeptide in the absence of the candidate compound.

The present invention also contemplates compounds which may exert their modulatory effect on SARS coronavirus polypeptides, such as 7a proteins by altering expression of the protein. In this case, such compounds may be identified by comparing the level of expression of the SARS coronavirus polypeptide in the presence of a candidate compound with the level of expression of the SARS coronavirus polypeptide in the absence of the candidate compound. SARS coronavirus polypeptides, such as 7a proteins and appropriate fragments and variants can be used in high-throughput screens to assay candidate compounds for the ability to bind to, or otherwise interact with these polypeptides.

It will be appreciated that the above described methods are merely examples of the types of methods which may be employed to identify compounds that are capable of interacting with, or modulating the activity of, the SARS coronavirus polypeptides, such as 7a, and fragments and analogues thereof, of the present invention. Other suitable methods will be known to persons skilled in the art and are within the scope of the present invention.

By the above methods, compounds can be identified which either activate (agonists) or inhibit (antagonists) activity of SARS coronavirus. Such compounds may be, for example, antibodies, low molecular weight peptides, nucleic acids or non-proteinaceous organic molecules.

SARS coronavirus function can be reduced or inhibited by the overexpression of certain members of the family of Bcl-2 related proteins. The family of Bcl-2-related proteins constitutes one of the biologically important gene products for the process of apoptosis, also called programmed cell death. Several members of this family are anti-apoptotic; i.e. over-expression inhibits apoptosis induced by many different stimuli (Gross et al. 1999; Vander Heiden and Thompson 1999; Tsujimoto and Shimizu 2000; Adams and Cory 2001). Examples of the anti-apoptotic members are Bcl-2, which was the first member of the family to be identified and was cloned from lymphoma cells carrying t(14;18) chromosomal translocations (Tsujimoto et al. 1985), and its functional homologue, Bcl-X_{L} (Boise et al. 1993).

Identification of host proteins interacting with SARS coronavirus 7a can be vital in elucidating possible functions of this unique viral protein. It can also provide crucial insight into the biology and pathogenicity of SARS coronavirus 7a. A yeast two-hybrid system to screen a B-lymphocyte cDNA expression library for cellular proteins capable of interacting with SARS-CoV 7a may be employed for identification of such host proteins. One principal cDNA identified from the screen encoded a ˜37.5-kDa peptide was identified as a human small glutamine-rich tetratricopeptide repeat (hSGT) containing protein.

The SARS coronavirus function may also be reduced or inhibited by antisense nucleic acids. The therapeutic or prophylactic use of such nucleic acids of at least six nucleotides, generally up to about 150 nucleotides, that are antisense to nucleic acids encoding SARS coronavirus polypeptides, such as 7a proteins. In this instance, these antisense nucleic acids refer to a nucleic acid capable of hybridising to at least a portion of SARS coronavirus RNA, such as 7a RNA (generally mRNA) by virtue of some sequence complementarity, and generally under high stringency conditions. Absolute complementarily is not required. Antisense nucleic acids in this form have utility as therapeutics that reduce or inhibit SARS coronavirus function, and can be used in its treatment or prevention.

The SARS coronavirus antisense nucleic acids may be of at least six nucleotides and are generally oligonucleotides which range in length from 6 to about 150 nucleotides. For example, the anti-sense oligonucleotide is at least 10 nucleotides, at least 15 nucleotides, at least 100 nucleotides, or at least 125 nucleotides. The oligonucleotides can be DNA or RNA or chimeric mixtures or derivatives or modified versions thereof, single-stranded or double-stranded.

The anti-sense oligonucleotide can be modified at any position on its structure with substituents generally known in the art. The SARS coronavirus oligonucleotide can include at least one modified base moiety which is selected from the group including, but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, pseudouracil, 2-thiocytosine, 5-methyl-2-thiouracil, 2-thiouracil, 4-thiouracil, 5-methyluracil, uracil-5-oxyacetic acid methylester, uracil-5-oxyacetic acid (v), queosine, wybutoxosine, 5-methyl-2-thiouracil, 3-(3-amino-3-N-2-carboxypropyl) uracil, and 2,6-diaminopurine.

In another aspect, the anti-sense oligonucleotide may include at least one modified sugar moiety, such as arabinose, 2-fluoroarabinose, xylulose, and hexose. The oligonucleotide may also include at least one modified phosphate backbone selected from a phosphorothioate, a phosphorodithioate, a phosphoramidothioate, a phosphoramidate, a phosphordiamidate, a methylphosphonate, an alkyl phosphotriester, and a formacetal or analog thereof.

The anti-sense oligonucleotide can be conjugated to another molecule, such as a peptide, hybridisation triggered cross-linking agent, transport agent or a hybridisation-triggered cleavage agent.

Expression of the sequence encoding the SARS coronavirus antisense RNA can be by any promoter known in the art to act in mammalian, including human, cells, and may include inducible or constitutive promoters. Examples of such promoters include: SV40 early promoter region (Bernoist and Chambon, Nature 290: 304-310 (1981), promoter in the 3′long terminal repeat of Rous sarcoma virus (Yamamoto et al., Cell 22: 787797 (1980), herpes thymidine kinase promoter (Wagner et al., Proc. Natl. Acad. Sci. U.S.A. 78: 1441-1445 (1981), or the regulatory sequences of the metallothionein gene (Brinster et al., Nature 296: 39-42 (1982), the disclosures of which are incorporated herein by reference.

RNA interference (RNAi) (see, eg. Chuang et al. (2000) PNAS USA 97: 4985) can be employed to inhibit the expression of a gene encoding a SARS coronavirus polypeptides, such as 7a. Interfering RNA (RNAi) fragments, particularly double-stranded RNAi, can be used to generate loss-of 7a function. Methods relating to the use of RNAi to silence genes in organisms are known, for instance, Fire et al. (1998) Nature 391: 806-811; Hammond, et al. (2001) Nature Rev, Genet. 2: 110-1119; Hammond et al. (2000) Nature 404: 293-296; Bernstein et al. (2001) Nature 409: 363-366; Elbashir et al (2001) Nature 411: 494-498; International PCT application No. WO 01/29058; and International PCT application No. WO 99/32619), the disclosures of which are incorporated herein by reference.

Role in Apoptosis

7a is the first ORF of subgenomic RNA7 and contains a signal peptide at the N terminus and a typical ER retrieval motif, KRKTE, at the C terminus. Induction of apoptosis in infected cells can contribute directly to the viral pathogenesis while inhibition of apoptosis can prevent premature death of the infected cells, allowing the virus to replicate to a high titer or allowing the establishment of a persistent infection.

In the case of SARS-CoV, studies on the clinical features of the disease have revealed some common abnormalities like elevated lactate dehydrogenase, lymphopenia, thrombocytopenia, hypocalcemia as well as liver enzyme abnormalities. However, until the present invention it was not apparent as to whether apoptosis plays an important role during SARS-CoV infection. In order to determine whether 7a had a role in the induction of apoptosis during infection, the inventors examined the ability of this SARS-CoV specific protein to induce apoptosis in cell-lines derived from different organs, and this is described in more detail in Example 3 set out below.

Disease Treatment and Diagnosis

Compounds identified by the above methods may be useful as therapeutic agents, particularly in the inhibition of apoptosis associated with overexpression of the gene product 7a in SARS infection. These compounds find use, for example, in treating or preventing a disease state in a subject, by administering a therapeutically effective amount of such a compound to the subject. Accordingly, pharmaceutically useful compositions comprising modulators of SARS coronavirus activity are contemplated. Suitable compositions may be formulated according to known methods such as, for example, by the admixture of a pharmaceutically acceptable carrier and an effective amount of the modulator.

Modulator and inhibitor compounds and agents of the present invention may be administered as compositions either therapeutically or preventively. In a therapeutic application, compositions are administered to a patient already suffering from a disease, in an amount sufficient to cure or at least partially arrest the disease and its complications. The composition should provide a quantity of the compound or agent sufficient to effectively treat the patient.

The therapeutically effective dose level for any particular patient will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

In general, suitable compositions may be prepared according to methods which are known to those of ordinary skill in the art and accordingly may include a pharmaceutically acceptable carrier, diluent and/or adjuvant.

These compositions can be administered by standard routes. In general, the compositions may be administered by the parenteral (e.g., intravenous, intraspinal, subcutaneous or intramuscular) route.

The carriers, diluents and adjuvants must be “acceptable” in terms of being compatible with the other ingredients of the composition, and not deleterious to the recipient thereof. Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

The compositions of the invention may be in a form suitable for administration in the form of a formulation suitable for oral ingestion (such as capsules, tablets, caplets, elixirs, for example), in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

Kits

In accordance with the present invention, kits containing nucleic acids encoding SARS coronavirus polypeptides, such as 7a, or anti-SARS coronavirus polypeptide antibodies, such as 7a antibodies may be prepared. Such kits may be used, for example, to detect the presence of SARS coronavirus infection in a biological sample. Detection using such kits is useful for a variety of purposes, including but not limited to disease diagnosis, epidemiological studies and performing screening methods of the present invention.

Kits of the present invention comprising one or more anti-SARS coronavirus polypeptide antibodies may further comprise one or more control antibodies which do not react with SARS coronavirus polypeptides, such as 7a, or antibodies of the present invention. Additionally, kits may contain means for detecting the binding of an anti-SARS coronavirus polypeptide antibodies, such as 7a antibodies For example the one or more SARS coronavirus antibodies may be conjugated to a detectable substrate such as a fluorescent, radioactive or luminescent compound, an enzymatic substrate, or to a second antibody which recognizes the SARS coronavirus antibody and is conjugated to a detectable substrate.

Kits according to the present invention may also include other components required to conduct the methods of the present invention, such as buffers and/or diluents. The kits typically include containers for housing the various components and instructions for using the kit components in the methods of the present invention.

SARS Coronavirus 3b Protein

The present invention also relates to a SARS coronavirus group-specific polypeptide 3b and to the nucleic acid encoding the same. Accordingly, one aspect of the invention provides an isolated expressed SARS coronavirus group-specific polypeptide 3b.

The inventors have found that the SARS coronavirus protein 3b can induce cell death when overexpressed. Thus, the expression of SARS coronavirus protein 3b at a certain stage of SARS infection may result in a high degree of cell-death in the SARS-coronavirus infected cells.

Accordingly, the invention also provides methods for the screening of compounds or agents capable of inhibiting infection by a SARS coronavirus and capable of treating or preventing SARS coronavirus in a subject. The invention further provides a method for treating or preventing SARS coronavirus in a subject.

The method of identifying an agent which is an inhibitor of infection by a SARS coronavirus may comprise contacting a cell or cell extract with one or more candidate agents, determining whether there is a decrease in the activity of the SARS coronavirus group-specific polypeptide 3b, and thereby determining whether the agent is an inhibitor of a SARS coronavirus.

The method of identifying an agent suitable for use in the treatment or prevention of SARS coronavirus in a subject may comprise:
(a) obtaining a biological sample from the subject,
(b) contacting the sample with a candidate agent,
(c) determining whether there is a decrease in the activity of the SARS coronavirus group-specific polypeptide 3b, and
thereby determining whether the agent is suitable for use in the treatment of SARS coronavirus.

The method for treating or preventing SARS coronavirus in a subject may comprise administering to the subject a therapeutically effective amount of a compound identified by the methods described above. The treatment or prevention of SARS coronavirus in a subject involves the inhibition of cell death associated with the overexpression of the SARS coronavirus protein 3b. The inhibition of cell death is achieved by inhibiting the formation of a complex between the SARS coronavirus group-specific polypeptide 3b and one or more polypeptides, such as SARS coronavirus group-specific polypeptides.

The present invention will now be described with reference to specific examples, which should not be construed as in any way limiting the scope of the invention.

### EXAMPLES

### Example 1

### Characterisation of 7a SARS Coronavirus Protein

Viruses and Cells Lines.

African green monkey kidney fibroblast (Vero E6) cells (American Type Culture Collection, Manassas, Va.) were maintained in complete Dulbecco's modified Eagle medium (Gibco) containing 10% fetal calf serum (HyClone Laboratories), 100 U of penicillin per ml, and 100 μg of streptomycin (Sigma) per ml. SARS-CoV strain SIN2774 (21) was used to infect subconfluent Vero E6 plates at a multiplicity of infection of 0.1. Subsequently, cells were harvested at the desired cytopathic effect (CPE), and total proteins were extracted. IBV-infected Vero E6 cell lysates were used as negative controls as indicated.

### Raising Antibodies to 7a

The cDNA encoding amino acids 16 to 111 was cloned into pGEX-4T-1 and transformed into Escherichia coli BL21 (DE3) cells. These cells were induced to express 7a (aa 16 to 111) with IPTG (isopropyl-β-D-thioga-lactopyranoside) and allowed to grow for 4 h at 37° C. Glutathione transferase-fusion proteins were purified, and the preparation was injected into mice for raising polyclonal antibodies. After four injections, the mice were bled, and the sera were tested for reactivity to 7a. The antibodies showed specific reactivity to 7a expressed in Vero E6 cells infected with the SARS virus or transfected with a 7a expression construct (FIG. 2B and C).

### Construction of Plasmids and Mutations

cDNAs were cloned into pXJ40-3′HA (GLAXO Group, Institute of Molecular and Cell Biology, Singapore, Republic of Singapore) for the expression of untagged proteins in mammalian cells; all constructs were untagged. All forward primers used were designed to incorporate a Kozak sequence. To create untagged proteins, all reverse primers (with the exception of mutL14-18R) contained the translation stop codon. The full-length 366-bp SARS-CoV ORF7a was amplified by PCR by using forward primer 7aF1 (5′-CGGGATCCACCATGGGAATGAAAAT-3′) (SEQ ID NO:3) and reverse primer 7aR2 (5′-CCGCTCGAGTCATTCTGTCTT-3′) (SEQ ID NO:4) incorporating BamHI and XhoI endonuclease restriction sites (underlined), respectively. The amplicon was purified, digested, and cloned into the compatible restriction sites of the expression vector to form pXJU122. To mutate the 7a signal peptide cleavage site, the amino acids SCELY located at positions 14 to 18 were mutated to leucines LLLLL by a two-step PCR-directed mutagenesis approach using plasmid pXJU122 as a template. Briefly, the overlapping primer set consisting of forward primer mutL14-18F (5′-GTATTTACATTGTTGCTGCTACTTCACTATCAGGAG-3′) (SEQ ID NO: 6) and reverse primer mutL14-18R (5′-CCTGATAGTGAAGTAGCAGCAACAATGTAAATACAATC-3′) (SEQ ID NO: 7) containing the incorporated mutations (underlined), were used in combination with primers 7aF1 and 7aR2 to create amplicon 7a-L. The amplicon was purified, digested, and cloned in the vector to create pXJU122L. Plasmid pXJmatU122 consisting of residues 16 to 122 was constructed by using PCR with forward primer 7aF2, 5′-CGGGATCCATGGAGCTATATCACT-3′, (SEQ ID NO:5) and reverse primer 7aR2; the BamHI restriction site is underlined and the incorporated ATG is indicated in bold. To study the signal retrieval motif the lysine residues in the 3′-terminal amino acids KRKTE at positions 118 and 120 were mutated to glutamic acid residues. This was done by using PCR with forward primer 7aF1 and reverse primer 7aRK>E(5′-CCGCTCGAGTCATTCTGTCTCTCTCAAT-3′ (SEQ ID NO:8) ; the XhoI restriction site is underlined). This construct (pXJU122K>E) expresses the untagged retrieval mutant. All sequences were confirmed by DNA sequencing.

### In Vitro Transcription and Translation

A total of 0.5 μg of plasmid pXJU122 was transcribed and translated by using the TNT T7 coupled reticulocyte lysate system (Promega) in a 10-μl reaction mixture for 1.5 h at 30° C. [³⁵S]cysteine (>1,000 Ci/mmol; NEN) was used to label 7a, and samples were immunoprecipitated by using 7a-specific antibodies with protein A-Sepharose. Proteins were resolved on sodium dodecyl sulfate (SDS)-15% polyacrylamide gel electrophoresis (PAGE) gels and visualized by radioautography with Amplify reagent (Amersham).

### Transfection, Pulse-Chase Radiolabeling, and Immunoprecipitation

The transfection of recombinant plasmids was accomplished by using liposomes (Lipo-fectamine 2000 reagent; Invitrogen). Generally, for a 6-cm plate, 0.5 μg of plasmid DNA was used for transfection according to the manufacturer's protocol.

For pulse-chase experiments, 2.0 μg of plasmid DNA was used for transfection per 6-cm plate. At 6 h posttransfection, confluent Vero E6 cells were starved for 30 min in prewarmed depletion medium lacking methionine and cysteine. The depletion medium was replaced with medium containing 100 μCi of [³⁵S]methionine-cysteine mix per ml (EXPRE³⁵S³⁵S protein labeling mix; NEN) for 10 or 30 min. [³⁵S]cysteine (100 μCi; NEN) was used to supplement the [³⁵S]methionine-cysteine mix to enhance the labeling efficiency. Subsequently, the cells were washed and chased with complete Dulbecco's modified Eagle medium containing a5 mM concentration of unlabeled methionine and cysteine. Radio-immunoprecipitation assay (RIPA; 1×) buffer was used to extract proteins, and immunoprecipitation was done with protein A-Sepharose-coupled antibodies as described by Nguyen and Hogue (17). Proteins were resolved by SDS-15% PAGE, and gels were fixed and treated with Amplify fluorographic reagent (Amersham). Subsequently, gels were dried and visualized by radioautography. The amount of labeled 7a was quantified by using a Bio-Rad model GS-700 imaging densitometer with Bio-Rad Multi-Analyst version 1.02/Mac software (number of radioautographs quantified for each experiment, 2).

### Immunofluorescence of SARS-CoV-Infected and Transfected Cells

SARS-CoV-infected Vero E6 cells were grown on coverslips until they showed a CPE of 25%. The coverslips were fixed in acetone for 20 to 30 min on ice and then air dried before being stored at −20° C. Before use, the coverslips were fixed again in methanol at −20° C. and air dried. For transfected proteins, Vero E6 cells were grown on coverslips and transfected as described above. An immunofluorescence assay was performed at about 16 h posttransfection as described by Goh et al. (7). Briefly, the medium was removed, and the coverslips were fixed in methanol for 5 min at −20° C., after which the coverslips were lifted out and completely air dried. To decrease background staining, mouse anti-7a sera were adsorbed against fixed Vero E6 cells. Uninfected cells showed no background staining with absorbed mouse anti-7a (FIG. 2C). Mouse anti-7a was used at a dilution of 1:200, and all other antibodies were used at dilutions of 1:100. Fixed cells were incubated with the appropriate primary antibody combination of mouse anti-7a and rabbit anti-GS28 (Golgi marker; BD, Singapore, Republic of Singapore) or rabbit anti-Sec31 (intermediate compartment marker Sec31; HWJ Group, Institute of Molecular and Cell Biology, Singapore, Republic of Singapore). Following washing, cells were incubated with the secondary antibody combination of fluorescein isothicyanate (FITC)-conjugated goat anti-mouse and rhodamine (Rh)-conjugated antirabbit antibodies (Santa Cruz Biochemicals). When cells were double-labeled with mouse anti-7a and rat anti-GRP94, fixed cells were sequentially incubated with rat anti-GRP94 (ER marker; ITS Science and Medical, Singapore, Republic of Singapore) and FITC-conjugated anti-rat secondary antibody, followed by incubation with mouse anti-7a and subsequently in Rh-conjugated antimouse (Santa Cruz Biochemicals). This was done to minimize the cross-reaction of secondary antibodies with both the primary antibodies.

### Example 2

### Results and Discussion of 7a SARS Coronavirus Protein Characterisation 7a Expressed in SARS-CoV-Infected Cells

Analysis of the SARS-CoV protein translated from ORF7a reflects a 122 amino acid polypeptide (polypeptide 7a) containing a signal peptide at the N terminus, a transmembrane domain, and a retrieval signal at the C terminus (FIG. 2A).

To determine if 7a was expressed in SARS-CoV-infected Vero E6 cells, cells were infected with SARS-CoV (strain SIN2774) as described above. Total proteins were harvested from Vero E6 cells showing 25 and 50 to 75% CPE and subjected to Western blotting. No signal was detected in mock-infected cells or IBV-infected cells (FIG. 2B, lanes 1 and 5), indicating the specificity of the mouse antibody against 7a. By using the mouse anti-7a antiserum, the protein was only detected in SARS-CoV-infected cells at a CPE of 50 to 75% (FIG. 2B, lane 3) and in Vero E6 cells transfected with a 7a DNA construct (FIG. 2B, lane 4). Two bands of about 15.5 and 15.0 kDa were detected in SARS-CoV late-infected cells (lane 3, 75% CPE), but a larger band of ˜17.5 kDa and a band of ˜15.0 kDa were detected in transient transfected cells expressing untagged 7a (lane 4). In SARS-CoV-infected cells, the immature 7a may have been processed efficiently, so that the immature form was not observed in infected cells. There appeared to be an additional band slightly larger than the mature form, indicating an intermediate form only present in virus-infected cells. 7a was not detected in the early phase of infection (lane 2, 25% CPE), even when the total protein of the sample with a 25% CPE that was used for Western blotting and immunodetection was double that of the sample with a 75% CPE (data not shown).

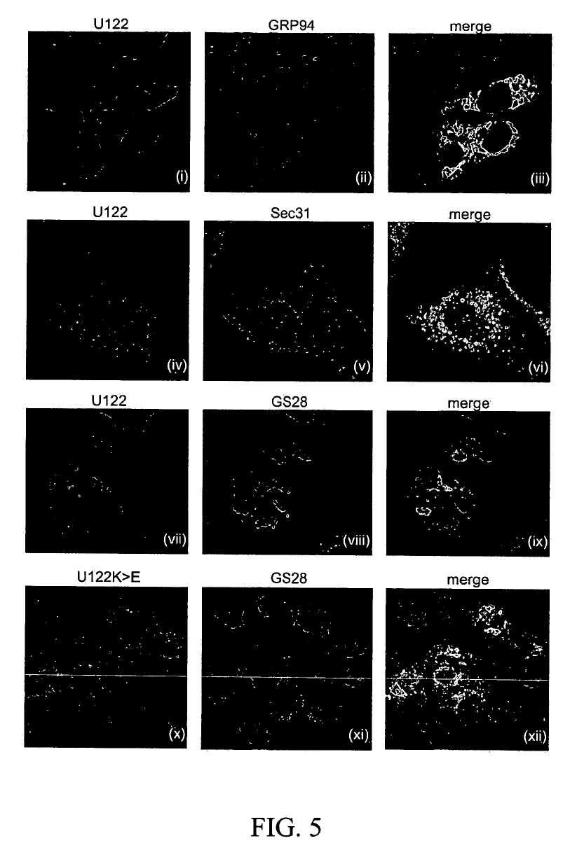

Immunofluorescence was used to determine the cellular localization of 7a in SARS-CoV-infected cells. By using mouse anti-7a antibody, the protein was detected in SARS-CoV-infected Vero E6 cells (FIG. 2C). 7a was detected in the perinuclear region and associated with ER. This cellular localization is similar to that observed in 7a-expressing Vero E6 cells (see FIG. 5).

### SARS-CoV 7a Protein in Transfected Cells

The SARS-CoV genome contains five group-specific ORFs larger than 50 amino acids (16, 20). SARS-CoV proteins, excluding the replication gene products of ORF lab, are translated from a set of 5′ nested subgenomic mRNAs (sgmRNAs). Each sgmRNA contains a 5′ end derived from the genomic 5′ leader sequence, subgenomic sequences, and a common 3′ end. Between five and eight SARS-CoV sgmRNAs are detected by Northern hybridization analysis from infected cells, ranging from 8.3 to 1.7 kb in size (20).

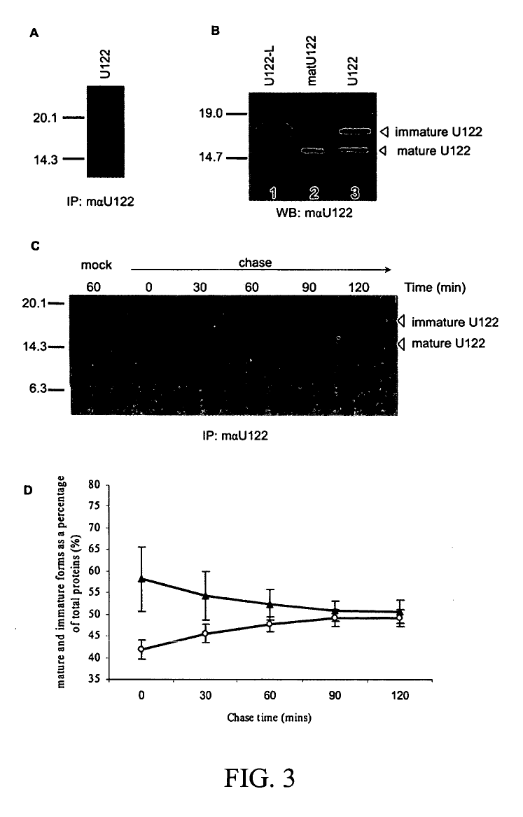

To express untagged SARS-CoV 7a in vitro, full-length ORF7a was cloned into mammalian expression vector pXJ40-3′HA to form pXJU122. Untagged 7a was translated in vitro by using the TNT coupled reticulocyte lysate system (Promega) in the presence of [³⁵S]cysteine. Following immunoprecipitation with antibodies specific for 7a, a single ˜17.5-kDa band was observed (FIG. 3A). To detect 7a expressed in Vero E6 cells, total proteins on Western blots were probed with mouse anti-7a. Two bands of about 17.5 and 15.0 kDa were observed (FIG. 3B, lane 3). The smaller protein band could be due to proteolytic cleavage of the signal peptide and was not observed in the in vitro translated product, even with the addition of canine microsomal membranes (data not shown), indicating the possible need for additional host cofactor(s) for efficient processing of 7a.

### The Putative SARS-CoV 7a Signal Sequence

Since the deduced SARS-CoV 7a amino acid sequence contains a putative signal peptide sequence of 15 residues, it would appear that the smaller ˜15.0-kDa product was due to cleavage of the signal peptide from the larger ˜17.5-kDa protein. Signal peptides play a major role in membrane integration and the translocation of secretory and membrane proteins from the ER. Many enveloped virus glycoproteins are synthesized as inactive precursors, which are usually unable to mediate membrane fusion and, hence, viral entry.

To determine whether the SARS-CoV signal peptide sequence is active, PCR-directed mutagenesis was used to create two mutations in 7a, one in the cleavage site of the putative signal peptide and the other to delete the N-terminal signal peptide up to the cleavage site. Mutation of the residues spanning the predicted cleavage site (residues SCELY at positions 14 to 18) to leucines (7a-L) abolished the cleavage of 7a in Vero E6-transfected cells (FIG. 3B, lane 1). The resultant ˜17.5-kDa product was similar in size to the untagged 7a immature product. Also, transient expression of the deletion mutant mat7a resulted in a product similar in size (˜15.0 kDa) to the mature form of the 7a peptide (FIG. 3B, lane 2). Pulse-chase analysis was done to determine the kinetics of wild-type untagged 7a expression in mammalian cells. 7a was expressed and radiolabeled in Vero E6 cells, and the radiolabeled 7a was immunoprecipitated. During the 2-h chase, the level of immature 7a slowly decreased, while the detectable level of mature 7a remained fairly constant (FIG. 3C). Expressed as a percentage of total 7a proteins, the immature form decreases, while the mature form increases through the 2-h chase (FIG. 3D), indicating that the uncleaved form was processed into the mature form. Both immature and mature forms could be detected from the start of the pulse-chase (FIG. 3C, time zero), indicating that the immature protein was cotranslationally processed to form the smaller mature protein. The total amount of labeled protein decreases, indicating that 7a is degraded in the course of the experiment. Combining these observations, the inventors considered that the constant level of the mature form may be due to the replacement of degraded mature protein by the conversion of immature protein to a mature form. Most of the conversion of immature to mature protein occurred cotranslationally (about 40%), and the remainder of the cleavage was posttranslational (about 10%). At the end of the 2-h pulse-chase, the processing was still not complete and reached a plateau of about 50% by 90 min, indicating that although posttranslational cleavage occurred, it was fairly inefficient.

### Mutation of the ER Retrieval Motif Leads to Rapid Proteolytic Processing of 7a

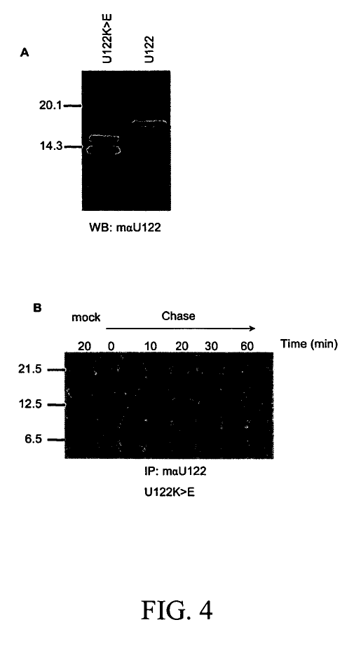

The short C-terminal tail of 7a contains a typical ER retrieval motif, KRKTE. In mammals, plants, and yeasts, this typical cytosolic C-terminal dilysine motif (KKXX or KXKXX, where X is any amino acid) is crucial for ER localization of type I membrane proteins (2). If the C-terminal X is considered the −1 position, the two lysine residues must be in the −3 and −4 or the −3 and −5 positions, respectively. These two lysine residues cannot be replaced by any other basic amino acid, and mutation of these residues results in the abolishment of the ER localization of the reporter proteins of mammalian cells. Studies have shown that the carboxyl-terminal sequence of Lys-X-Lys-X-X in integral ER membrane proteins functions either as a retrieval or retention signal, with signals important for the transport of these proteins back to the ER from the intermediate compartment (19). To determine whether this retrieval motif was functional, the lysine residues at positions 118 and 120 were mutated to glutamic acid by using PCR-directed mutagenesis (7aK>E). Transient expression of plasmid pXJU122K>E in Vero E6 cells was done to determine the effect of the mutation on protein expression and localization. Total proteins were harvested at 16 h post-transfection, subjected to Western blotting, and probed with mouse anti-7a (FIG. 4A). Two bands (˜15.5 and ˜12.5 kDa) were detected in the cells expressing 7a>E (FIG. 4A, lane 1), which were different in size from those observed for 7a. The processing of the immature form appears to be very efficient, as the larger band (17.5 kDa) is no longer visible. The two bands produced by the mutant untagged protein differed in size from the smaller band (15.0 kDa) of 7a. The mutations from K to E may cause the small change in mobility, or mutations in the recycling signal may influence the processing of the 7a at the N terminus so that the alternative cleavage sites are used. Pulse-chase analysis of 7a>E was performed to verify the efficiency of this processing. Following a 10-min pulse-labeling, the immature ˜17.5-kDa product was not detected at the start of the chase, indicating that the immature product has been converted cotranslationally to two smaller products of ˜12.5 kDa (FIG. 4B). Benghezal et al. (2) observed similar results with green fluorescent protein-fusion proteins fused to mutated retrieval signals in plants. They suggested that the mutated retrieval signal caused rapid protein exit from the ER to a distal compartment where the processing activity is located. The larger band of the doublet is eventually converted to the smaller ˜12.5-kDa product after the 60-min chase, suggesting an additional proteolytic cleavage site in the K>E mutant (FIG. 4B).

### SARS-CoV 7a is Localized to the ER and ER-Golgi Intermediate Compartment in Transfected Cells

The subcellular localization of 7a in Vero E6 cells was studied. At 16 h posttransfection, cells were fixed with methanol and stained with both mouse anti-7a antibodies (FIG. 5, left frames) and antibodies to either the ER marker GRP94 (FIG. 5, ii), the ER intermediate compartment marker Sec31 (frame v) or the Golgi marker GS28 (frames viii and xi). 7a was observed to colocalize with GRP94 (FIG. 5, iii), indicating that 7a was present in the ER compartment. In cells showing weaker 7a expression, 7a and Sec3 1-labeled punctate structures could be detected. These probably corresponded to the ER-Golgi intermediate compartment (FIG. 5, vi). 7a did not colocalize with the cis-Golgi marker GS28 (FIG. 5, ix). This is likely due to the rapid movement of 7a from the Golgi compartment back to the ER, as seen in recycled proteins with retrieval signals (6). On the other hand, mutant 7aK>E was predominantly localized to the Golgi (FIG. 5, xii). Following cleavage of the signal sequence, it is rapidly transported to the Golgi but cannot be recycled back to the ER because of the mutated retrieval sequence, resulting in a large fraction of 7aK>E remaining in the Golgi apparatus. Collectively, immunofluorescence results indicated that untagged 7a was cycled between the ER and Golgi compartments and that this process was mediated by the retrieval motif at the C terminus.

Interestingly, convalescent-phase human sera could not detect bacterially expressed 7a-glutathione transferase fusion protein by Western blot analysis (25). This suggests that 7a is either not a viral structural protein or that it is not exposed or sufficiently immunogenic in vivo. Importantly though, 7a has been shown to interact specifically with another unique group-specific SARS-CoV protein (ORF3a on sgRNA 3), designated 3a (FIG. 1). The latter has been shown to interact with the SARS-CoV structural proteins E, S, and M (24). Also, in this study 7a was shown to localize to the intermediate compartment, where CoVs are known to assemble and bud (12).

### Example 3

### Overexpression of 7a SARS Coronavirus Protein and Apoptosis

In order to determine if 7a played any role in the induction of apoptosis during infection, the inventors examined the ability of this SARS-CoV specific protein to induce apoptosis in cell-lines derived from different organs.

In doing so, 293T cells where transfected with cDNA constructs for expressing the various SARS-CoV genes using lipofectamine reagent (Invitrogen, Carlsbad, Calif., USA). These constructs were obtained from SARS CoV 2003VA2774, an isolate from a SARS patient in Singapore (11, 27, 28). 2 μg of DNA was used to transfect 1×10⁶ cells and the cells were left for approximately 16 h. When HA-GST (HA tagged glutathione S-transferase protein) was expressed, it was observed that the expression level of this protein was extremely high, therefore, 0.25 μg of the plasmid for expressing HA-GST together with 1.75 μg of empty vector were used instead. The cells were harvested and washed with PBS and then resuspended in ˜100 μl of hypotonic cell lysis buffer. The suspension of cells was divided into 2 aliquots, one to be used for measuring caspase-3 protease activity and one for Western blot analysis.

One aliquot was subjected to 5 rounds of freeze-thaw cycles and then centrifuged to remove the cell debris. The total protein concentration in the lysate was determined using Coomassie Plus reagent from Pierce (Rockford, Ill., USA). The amount of caspase3 protease activity in 10 μg of total protein was then determined using CaspACE fluorometric assay system from Promega Corporation (Madison, Wis., USA). Laemmli's SDS buffer was added to the other aliquot which was then heated at 100° C. for 10 min and then 20 μl of this total cell suspension was subjected to Western blot analysis. For detection of endogenous poly (ADP-ribose) polymerase (PARP) protein, a polyclonal antibody (Cell Signalling Technology Inc. Beverly, Mass., USA) that recognizes full-length PARP (116 kDa) and the cleaved form of PARP (83 kDa), was used.

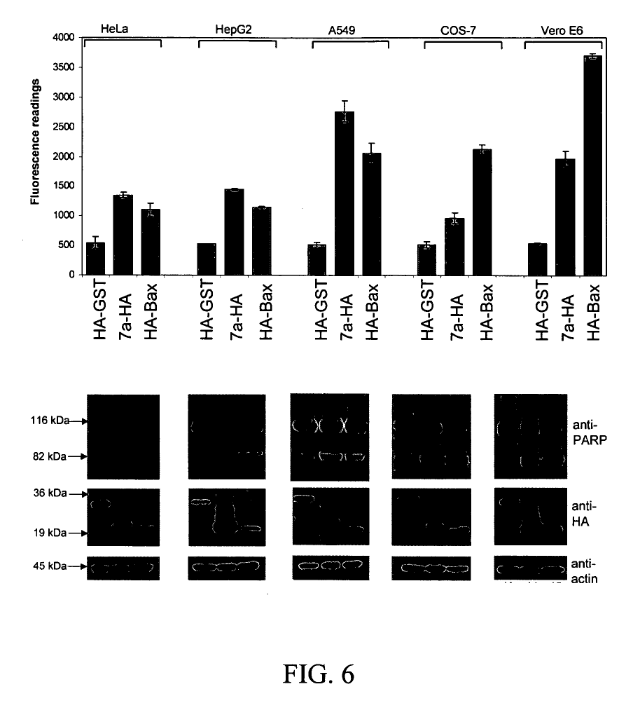

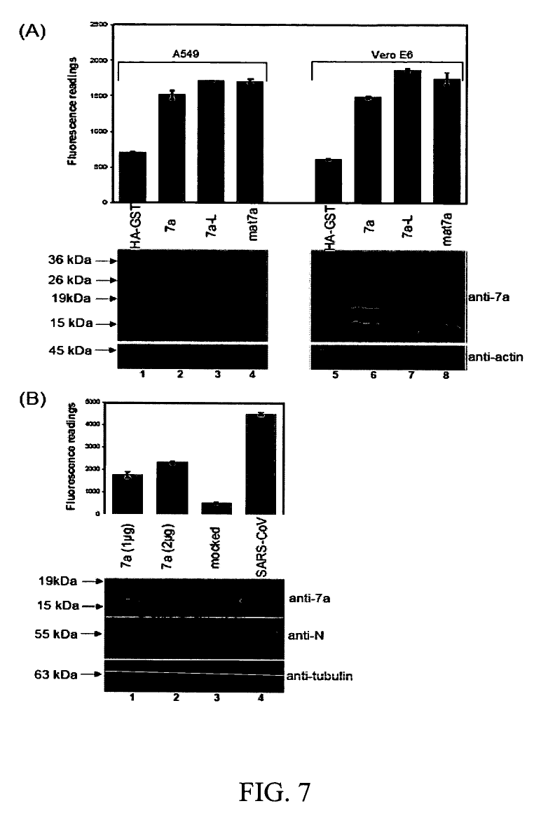

The over-expression of 7a-HA induces apoptosis in 293T (human kidney epithelial) cells as evident by an increase in caspase-3 protease activity, a hallmark of apoptosis, which is comparable to that caused by the over-expression of BAX, a pro-apoptotic member of the Bcl-2 family (FIG. 6A, columns 1 and 3). Cleavage of endogenous PARP was also observed when either 7a-HA or HA-BAX was over-expressed (FIG. 6B, lanes 1 and 3). On the other hand, 3a-HA did not significantly induce apoptosis, neither did the structural proteins, M-HA, E-HA and HA-N (FIG. 6A, B, lanes 4 to 7). To determine the degree of cell death, cell viability was also determined using the cell proliferation reagent, WST-1 (Roche Molecular Biochemicals, Indianapolis, Ind., USA). In this case, 0.3×10⁶ cells in a 24-well plate were transfected with 0.35 μg of DNA and then assayed after 16 h and the percentage of live cells are shown in FIG. 7A in parentheses. Consistent with the caspase assay, overexpression of 7a leads to cell death as evident from the decrease in cell viability to ˜44% (FIG. 6 1A, column 3).

Apoptosis was also observed with the expression of either the HA-tagged 7a, or the untagged form of 7a and was mediated via caspases since it was strongly blocked by the pan-caspase inhibitor z-VAD-fmk (FIG. 6C, D, lanes 1 to 4). In contrast, no inhibition of apoptosis was observed in the presence of an irrelevant peptide, z-FA-fink (FIG. 6C, D, lanes 5 to 7). About 50% of the precursor form of 7a (˜17.5 kDa) is cleaved after the signal peptide at the N terminus yielding a product of ˜15 kDa (FIG. 6D, lanes 3 and 6). However, the HA-tagged form of 7a does not appear to undergo cleavage as only a single band of ˜22 kDa, which corresponds to unprocessed form of 7a
(17.5 kDa) fused with 3 HA (YPYDVPDYA) motifs (˜4 kDa) at the C terminus, is observed (FIG. 6D, lanes 2 and 5).

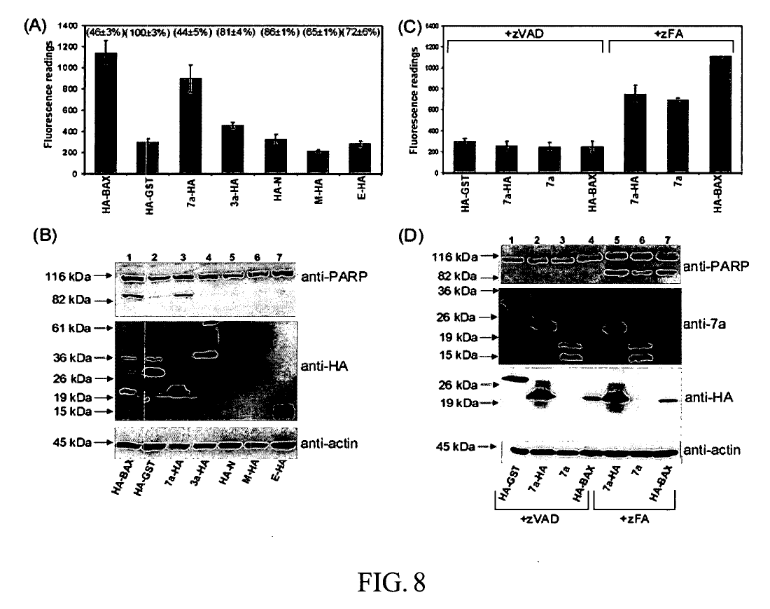

The experiment was repeated with cell-lines derived from different organs (FIG. 7): cervical (HeLa, human cervical carcinoma), lung (A549, human lung carcinoma), liver (HepG2, human hepatocellular carcinoma), and kidney (Vero E6 and COS-7, African green monkey kidney epithelial and fibroblast respectively). Increases in capase3 protease activities and PARP cleavage were observed when 7a-HA (FIG. 7, lanes 2, 5, 8, 11, 14) or HA-BAX (FIG. 7, lanes 3, 6, 9, 12, 15) were over-expressed in all the cell-lines tested, in comparison to cell transfected with a control plasmid, HA-GST (FIG. 7, lanes 1, 4, 7, 10, 13). The signal peptide at the N terminus of 7a is cleaved more efficiently in infected cells than in transfected cells, but this cleavage is not important for apoptosis induction as both mutants 7a-L, which contains mutations at the cleavage site, and mat7a, where the signal peptide has been deleted, induce the same degree of apoptosis as wild-type 7a (FIG. 8). This result is also consistent with the data shown in FIG. 6C and 6D where both 7a and 7a-HA, where the signal peptide is not cleaved, induce similar degree of apoptosis.

The levels of caspase-3 protease activity in lysates obtained from Vero E6 cells transfected with 7a cDNA (FIG. 8B, lanes 1 and 2) are significantly lower than that for SARS-CoV infected Vero E6 cells (FIG. 8B, lane 4), even though the expression levels of 7a are comparable (FIG. 8B). These cells were infected at multiplicity of infection of 0.1 and harvested 24 h post infection.

This data suggests that 7a of SARS-CoV is an example of a coronavirus protein that can induce apoptosis when over-expressed. The ability of 7a to induce apoptosis in different cell-types is consistent with the clinical observation of apoptosis in different organs infected by SARS-CoV and suggests that the expression of 7a during infection may be one of the underlying mechanisms for the pathogenesis of SARS-CoV infection.

### Example 4

### Overexpression of BC1-X_{L} Inhibits Apoptosis by SARS Corona Virus 7a Protein

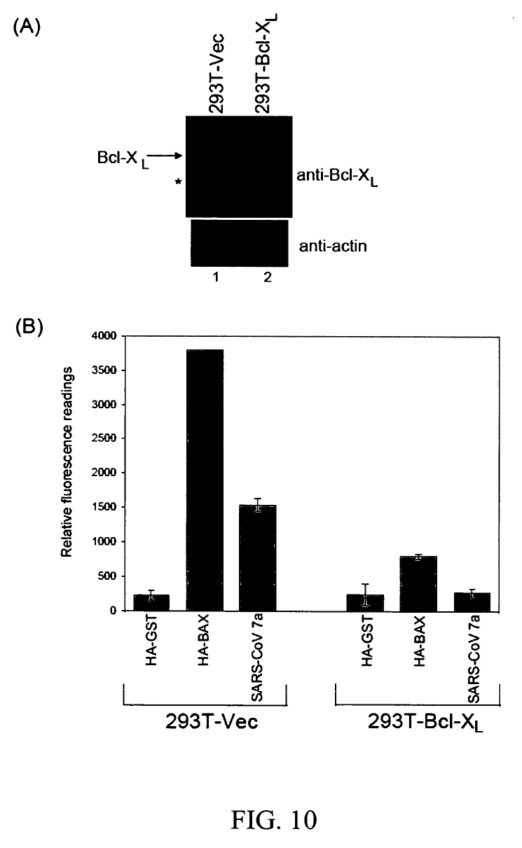

In order to determine if SARS-CoV 7a acts upstream or downstream of the Bcl-2 family in the apoptotic pathway, the Bcl-X_{L} gene was cloned into the pCep4 vector (Invitrogen) and transfected into 293T cells. Cells stably expressing Bcl-X_{L} were obtained after antibiotic selection as previously described (Tan et al., 2003) and the expression of Bcl-X_{L} was analyzed by Western blot analysis using a specific anti-Bcl-X_{L} antibody. Control cells were stably transfected with an empty vector. As shown in FIG. 10A, there was high expression of Bcl-X_{L} in the chosen stable clone (293T-BC1-X_{L}, clone 10A) when compared to the low endogenous level in the vector control (293T-Vec, clone 1A).

These stable clones were then transiently transfected with cDNA to express HAGST (negative control), HA-BAX (positive control) and SARS-CoV 7a as previously described (Tan et al., 2004a). As shown in FIG. 10B, the expression of HA-BAX and SARS-CoV 7a resulted in apoptosis in 293T-Vec cells (columns 1-3) as determined by a fluorometric assay for measuring caspase activation (Promega). However, in the 293TBcl-X_{L} cells, the expression of HA-BAX and SARS-CoV 7a gave a significantly lower degree of apoptosis (FIG. 10B, columns 4-6). Thus, the overexpression of Bcl-X_{L} inhibited the induction of apoptosis by SARS-CoV 7a, suggesting that the induction of apoptosis by SARS-CoV 7a occurs at or upstream of the Bcl-2 family in the apoptotic pathway.

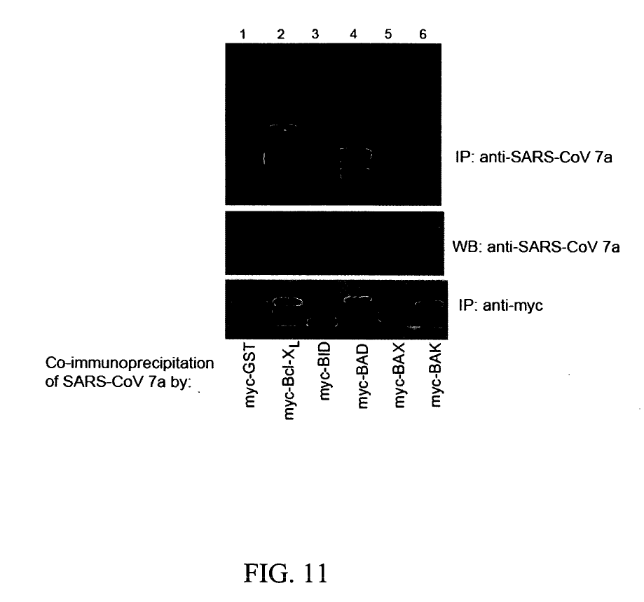

To further understand how the SARS-CoV 7a may modulate the function of the Bcl-2 family of proteins, co-immunoprecipitation experiments were performed to determine whether SARS-CoV 7a interacted with members of the Bcl-2 family. As shown in FIG. 11 (1ₛₜ panel), myc-Bcl-X_{L} and myc-BAD specifically co-immunoprecipitated SARS-CoV 7a (lanes 2 and 4). This was not observed for the other members tested (myc-BID, myc-BAX or myc-BAK) or the negative control (myc-GST) (lanes 1, 3, 5 and 6). These results indicate the SARS-CoV 7a protein may induce apoptosis by interfering with the function of the Bcl-2 family proteins, in particular, Bcl-X_{L} and BAD.

### Example 5

### Small Glutamine-Rich Tetratricopeptide Repeat-Containing Protein (SGT) Interacts with SARS-CoV 7a

### Mammalian Cell Lines, DNA Constructs and Antibodies

African green monkey kidney epithelial (Vero E6) cells (American Type Culture Collection, Manassas USA) were maintained as described previously (Fielding et al., 2004). cDNA clones expressing full length (HA tagged and untagged) and deletion mutants of 7a (HA tagged) were prepared as described previously (Fielding et al., 2004; Tan et al., 2004). Full length as well as truncations of hSGT cDNA, tagged with a N-terminus flag epitope for expression in mammalian cells are summarized in Table 1 below.

TABLE 1
Plasmids used in this study

| Plasmid | Description | Source |
|---|---|---|
| pAS2-1 | Gal4 DNA-binding domain in a 2 μm | Clontech |
| | TRP1 yeast shuttle vector | Inc. |
| pAS2-1-7aΔ96-122 | Deletion of C-terminus | This work |
| | (ORF7a aa 1-95) | |
| pACT2 | Gal4 activation domain in a 2 μm | Clontech |
| | LEU2 yeast shuttle vector | Inc. |
| pXJ40-myc | Mammalian expression for tagging | V. Yu lab, |
| | proteins with c-myc at the N-terminus | IMCB* |
| pXJ40-flag | Mammalian expression for taggings | Manser et |
| | proteins with flag epitope at the | al., 1997 |
| | N-terminus | |
| pXJ40-3′HA | Mammalian expression for tagging | GLAXO |
| | proteins with HA at the C-terminus | lab, |
| | | IMCB* |
| pXJ40-flagSGT | Full length SGT, flag tagged at | This work |
| | N-terminus | |
| pXJ40-flagSGTΔC | Deletion of C-terminus | This work |
| | (SGT aa 1-192) | |
| pXJ40-flagSGTΔC-3 | Deletion of C-terminus and TPR-3 | This work |
| | (SGT aa 1-158) | |
| pXJ40-flagSGTΔC2-3 | Deletion of C-terminus and TPR-2 | This work |
| | and -3 (SGT aa 1-124) | |
| pXJ40-flagSGTΔN-1 | Deletion of N-terminus and TPR-1 | This work |
| | (SGT aa 125-313) | |
| pXJ40-flagSGTΔN1-2 | Deletion of N-terminus and TPR-1 | This work |
| | and -2 (SGT aa 159-313) | |
| pXJ40-7a-HA | Full length ORF7a, HA tagged at | Tan et al., |
| | C-terminus | 2004c |
| pXJ40-7aΔC-HA | Deletion of C-terminus | This work |
| | (ORF7a aa 1-61) | |
| pXJ40-7aΔN-HA | Deletion of N-terminus | This work |
| | (ORF7a aa 62-122) | |

*IMCB, Institute of Molecular and Cell Biology, Singapore

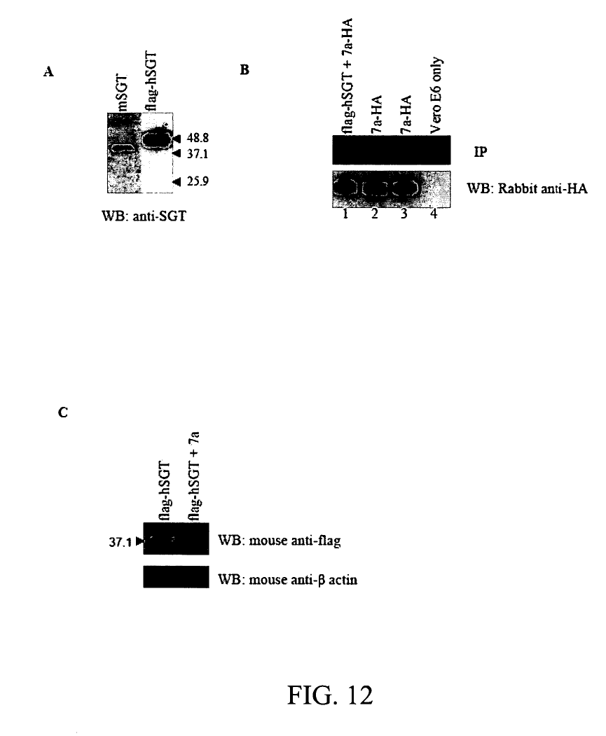

Polyclonal anti-human SGT was a generous gift from Dr A. T. Panganiban (Wisconsin, USA). The rabbit anti-SGT antibody specifically detected endogenous SGT from Vero E6 cells as well as transfected flag-SGT (FIG. 12A). Polyclonal and monoclonal anti-HA (Santa Cruz) and anti-flag (Sigma) antibodies were used according to the manufacturer's instructions.

### Two-Hybrid System Library Screen

The yeast reporter strain AH109 [GAL4 2H-3] (Clontech) was used for the two-hybrid selection. Plasmid pAS2-7aΔ96-122 was used as bait and a pACT-cDNA library (human lymphocyte MATCHMAKER, Clontech) was used as the source of prey genes. Yeast cells were grown on YPD or on synthetic minimal medium (0.67% yeast nitrogen base, the appropriate auxotrophic supplements, 2% agar [for plates]) supplemented with 2% dextrose. Yeast was transformed with appropriate plasmids by the lithium acetate method and transformants were selected on synthetic minimal medium. The bait plasmid and the pB42AD cDNA library were introduced into the yeast strain AH109 [GAL4 2H-3]. Two-hybrid screen and interaction assays were performed essentially as described in the protocol (Clontech) in the presence of 2% galactose and 80 mg of 5-bromo-4-chloro-3-indolyl-Dgalactopyranoside per liter. Prey plasmids were selected from yeast colonies giving a positive signal according to the manufacturer's protocol. False positives were eliminated by retransforming the host AH109 [GAL4 2H-3] strain with pACT-cDNA library plus bait plasmid. Also, the pACT-cDNA was transfected in yeast strain PJ69-2A to check for autoactivation. The positive clones that contained cDNAs encoding 7a-interacting proteins were sequenced and analysed using BLAST.

### Transient Expression and Western Blotting

Typically, 1 μg of plasmid cDNA was used for transfection into Vero R6 cells in transient expression studies; full length flag-hSGT was used at 0.25 μg. About 16 hours after transfection, radio-immunoprecipitation (RIPA) buffer was used to extract total proteins and cell lysates were separated by 15% SDS-polyacrylamide gel electrophoresis and transferred to nitrocellulose membranes (Amersham). Membranes were blocked in blocking solution (3% nonfat dry milk and 0.05% Tween 20 in PBS) and incubated in the appropriate primary antibody for at least 16 hrs at 40° C. with rolling. Following extensive washing with 0.05% Tween 20 in PBS, the membranes were incubated in the appropriate secondary antibody for 1 hr at room temperature. After washing, membranes were developed with an enhanced chemiluminescence kit (Amersham).

### Immunoprecipitation

Transfected Vero E6 cells were washed twice with ice cold phosphate buffered saline (PBS) and cells were solubilised for 10 min at 40° C. in lysis buffer (ingredients). Insoluble cell material was removed by centrifugation at 14000 rpm for 10 min at 40° C. Typically, 200 μg of whole cell lysates were incubated with mouse anti-flag beads or rabbit anti-SGT antibody conjugated to protein A-agarose beads for 16 hours at 40° C. with end-over-end mixing. Following incubation, the beads were collected and complexes were washed three times with lysis buffer. The bound proteins were eluted by boiling in SDS sample buffer and Western Blotted as discussed elsewhere.

### Sequencing of the African Green Monkey Kidney Epithelial SGT

Total cellular RNA was extracted from Vero E6 cells using the RNeasy Mini Kit (Qiagen) according to the manufacturer's instructions. First-strand cDNA was prepared from 1.0 μg total RNA using the SuperScript II RNase Reverse Transcriptase kit (Invitrogen). Subsequently, a 1:10 dilution of the first-strand cDNA was used in a PCR reaction according to standard protocols. The primary nucleotide sequence of African green monkey kidney epithelial SGT was determined by automated sequencing and compared to the hSGT sequence (NCBI accession number NP_{—}003012) using CLUSTAL X. The conceptual amino acid sequence of mSGT was compared to hSGT and comparisons were visualized using GENEDOC software.

### Immunoflourescence

Transiently transfected Vero E6 cells were grown on coverslips and transfected as above. At about 16 h post-transfection, the medium was removed and the coverslips fixed in methanol at −20° C. After 5 min the coverslips were lifted out and completely air-dried. Fixed cells were incubated with the primary antibody combination of mouse anti-HA and rabbit anti-flag at room temperature for 1 hr. Mouse anti-HA and rabbit anti-flag antibody were used at a dilution of 1:200. Following washing, cells were incubated with the secondary antibody combination of FITC-conjugated goat anti-rabbit and Rh-conjugated anti-mouse antibodies at room temperature for 1 hr (Santa Cruz Biochemicals, USA). Following extensive washing, the coverslips were mounted on glass slides and viewed.

### Results and Discussion

SGT interacts with SARS-Co V 7a in Vero E6 Cells

Co-immunoprecipitation studies were used to confirm the interaction between hSGT and 7a-HA in Vero E6 cells. Cells were co-transfected with pXJ40-flaghSGT and pXJ40-7aHA, as described elsewhere. For 7a-HA interaction with endogenous Vero E6 SGT, only pXJ40-7aHA was transfected. Total protein extracts were immunoprecipitated with mouse anti-flag beads or rabbit anti-hSGT antibody respectively and Western Blotted. Western Blots were detected with rabbit anti-flag or rabbit anti-HA (FIG. 12B). SARS-CoV 7a-HA coimmunoprecipitated with both flag-hSGT (lane 1), as well as endogenous SGT (lane 2) from Vero E6 cells. 7a-HA was not detected when an unrelated antibody (lane 3) or untransfected cells (lane 4) were used for immunoprecipitation. Interestingly, in Western blot analysis of total proteins from cells transfected with pXJ40-flaghSGT and pXJ40-7a, it was noted that flag-hSGT was degraded (FIG. 12C). This phenomenon was only observed when untagged 7a was used in Vero E6 cells. Similarly, flag-SGT was degraded when Bax-HA was overexpressed in VeroE6 cells. This degradation was blocked by the addition of the pancaspase inhibitor zVAD-fink, but not in the presence of an irrelevant peptide, zFA-fmk (data not shown). The latter indicated that the degradation of flag-SGT could be a result of apoptosis induced by Bax-HA and 7a.

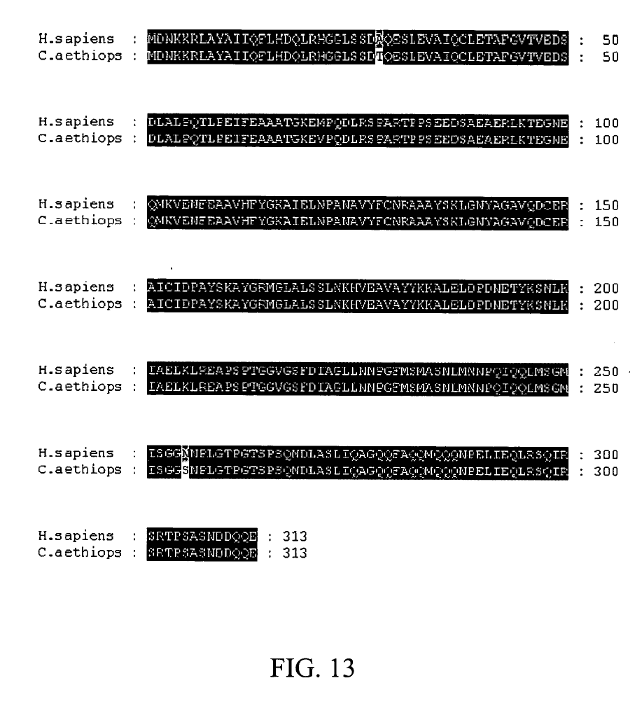

To investigate the interaction of 7a with both human SGT as well as African green monkey SGT (mSGT), the inventors determined the primary nucleotide sequence of mSGT using cDNA from Vero E6 cells. The deduced primary sequence was compared to hSGT using CLUSTAL X (Thompson et al., 1999) and visualized with GENEDOC (Nicholas and Nicholas, 1997). mSGT showed >96% nucleotide and >99% amino acid sequence identity with hSGT (FIG. 13). This explained why 7a interacted with SGT from two distinct organisms.

SARS-CoV 7a Co-Localizes with Flag-hSGT

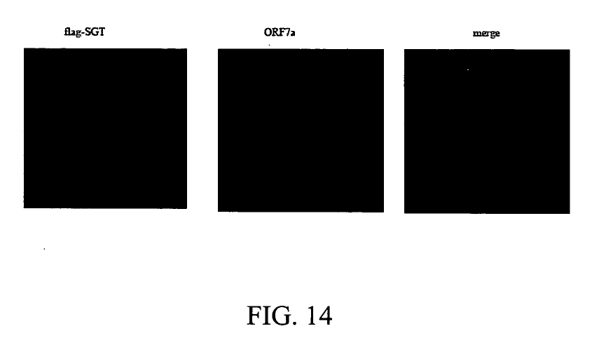

The sub-cellular localization of 7a-HA and flag-hSGT in Vero E6 cells was studied. Vero E6 cells were transfected with pXJ40-flagSGT and pXJ40-7aHA (FIG. 14). At 16 hrs post-transfection, cells were fixed with methanol and stained with both mouse anti-HA and rabbit anti-flag antibodies. As a negative control untransfected Vero E6 cells were treated in the same way. Results showed that flag-SGT was distributed to both the nucleus and cytoplasm of transfected cells. Untransfected cells did not stain with the antibodies. 7a-HA was found to co-localize with flagSGT in Vero E6 cells. Cziepluch and co-workers (1998) also reported that untagged rat SGT was detectable in cytoplasm and nucleus of FREJ4 cells. This co-localization of flag-hSGT and 7a-HA indicates that SARS-CoV 7a may interact with SGT in Vero E6 cells.

The C-Terminal Region (aa 62-95) of SARS-CoV 7a is Essential for Interaction with hSGT

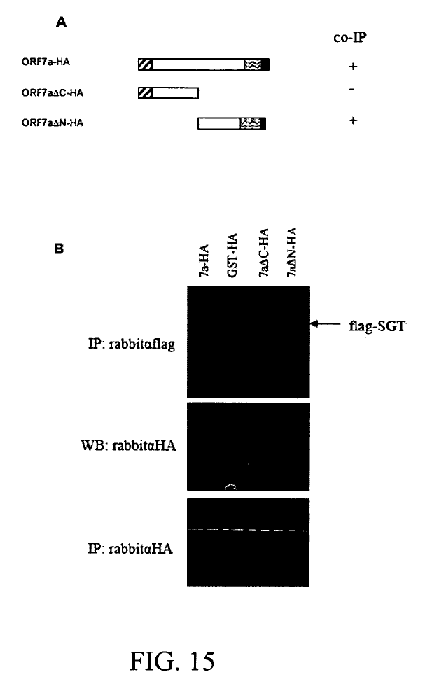

The initial results using yeast two-hybrid and immunopreciptation experiments indicated that the 7a mutant pAS2-1-7aΔ96-122, as well as full length 7a interacted with hSGT. To identify the region of interaction, a series of deletion mutants of HA-tagged SARS-CoV 7a were constructed (FIG. 15A). Interaction of flag-hSGT and 7a-HA deletion mutants was assayed using co-immunoprecipitation in Vero E6 cells. Results showed that 7a-HA (aa 1-122) and ORF7aΔNHA (aa 62-122) interacted with flag-SGT, but 7aΔC-HA (aa 1-61) did not (FIG. 15B). Also, since pAS2-7aΔ96-122 interacted with full length 7a in the yeast two hybrid screens, the region of interaction was located within aa 62-95.

hSGT TPR2 is Critical for the Association with SARS-CoV 7a

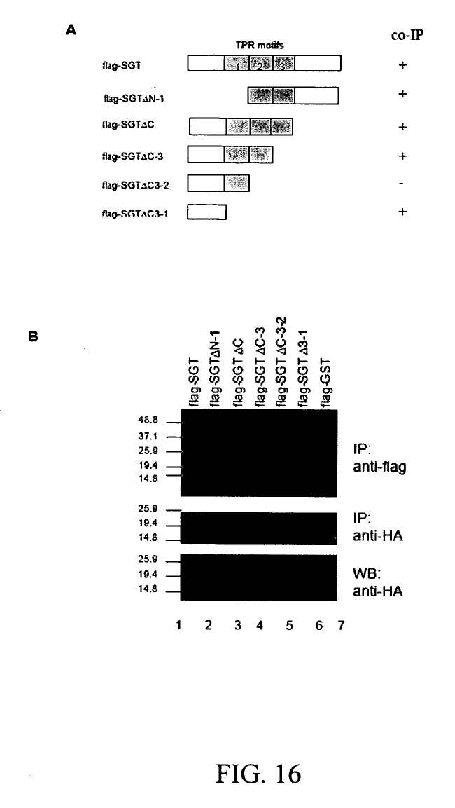

hSGT contains three TPR 34 aa motifs that have been shown to mediate protein protein interaction. Therefore, to determine whether these motifs play a role in the interaction between hSGT and SARS-CoV 7a, five hSGT deletion mutants were created (FIG. 16A). Binding of the hSGT mutants with 7a was studied using immunoprecipitation assays in Vero E6 cells. Results showed that full length flag-SGT (aa 1-313), flag-SGTΔN-1 (aa 125-313), flag-SGTΔC (aa 1-192), flag-SGTΔC-3 (aa 1-158) and flag-SGTΔC3-1 (aa 1-91) interacted with 7a-HA. However, flag-SGTΔN1 reacted relatively weaker than the other positives. On the other hand, flag-SGTAC3-2 (aa 1-124) did not interact with 7a at all (FIG. 16B). Thus, only hSGT mutants that contained TPR2 (aa 125-158) interacted with 7a and the N terminus region somehow improved the interaction efficiency. These results indicated that TPR2 was essential, but not the only motif, for interaction with SARS-CoV 7a.

### Example 6

### Overexpression of 3b SARS Coronavirus Protein and Apoptosis

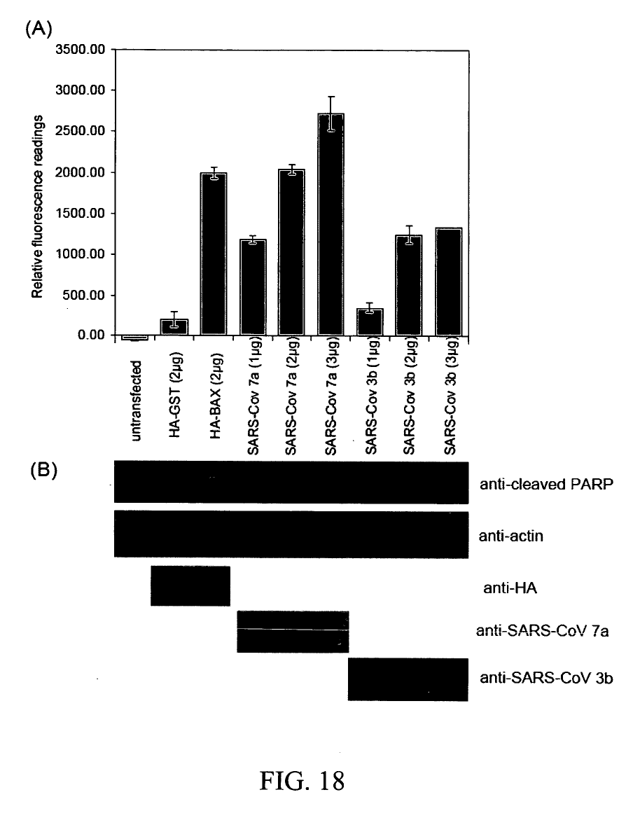

The inventors examined the ability of SARS coronavirus 3b protein to induce apoptosis when it is overexpressed in 293T cells. The 3b gene was obtained from isolate SIN2774 and cloned into a pXJ3′ vector as described in Tan et al., 2004b. As shown in FIG. 18A, the overexpression of SARS-CoV 3b protein induced apoptosis as evident by an increase in caspase-3 protease activity (columns 7 to 9), which is comparable to that caused by the over-expression of HA-BAX (column 3) or SARS-CoV 7a (columns 4 to 6). Cleavage of endogenous PARP to a 83 kDa cleaved form was also observed when either HA-BAX, SARS-CoV 7a or SARS-CoV 3b was overexpressed (FIG. 18B, 1st panel).

### Example 7

### Compositions for Treatment

The inhibitor compounds identified by the methods of the present invention, or the apoptotic 7a polypeptide useful for the treatment or prevention of disease states may be administered alone, although it is preferable that they be administered as a pharmaceutical composition.

In accordance with the best mode of performing the invention provided herein, specific preferred compositions are outlined below. The following are to be construed as merely illustrative examples of compositions and not as a limitation of the scope of the present invention in any way.

### Example 7(a)

### Composition For Parenteral Administration

A composition for intramuscular injection could be prepared to contain 1 mL sterile buffered water, and 1 mg of a suitable inhibitor or compound.

Similarly, a composition for intravenous infusion may comprise 250 ml of sterile Ringer's solution, and 5 mg of a suitable inhibitor compound.

### Example 7(b)

### Injectable Parenteral Composition

A composition suitable for administration by injection may be prepared by mixing 1% by weight of 7a polypeptide in 10% by volume propylene glycol and water. The solution is sterilised by filtration.

### Example 7(c)

### Capsule Composition

A composition of an inhibitor compound in the form of a capsule may be prepared by filling a standard two-piece hard gelatin capsule with 50 mg of the inhibitor compound, in powdered form, 100 mg of lactose, 35 mg of talc and 10 mg of magnesium stearate.

### Example 7(d)

### Composition For Inhalation Administration

For an aerosol container with a capacity of 20-30 ml: a mixture of 10 mg of a inhibitor compound with 0.5-0.8% by weight of a lubricating agent, such as polysorbate 85 or oleic acid, is dispersed in a propellant, such as freon, and put into an appropriate aerosol container for either intranasal or oral inhalation administration.

### REFERENCES

1. Anand, K., J. Ziebuhr, P. Wadhwani, J. R. Mesters, and R. Hilgenfeld. Coronavirus main proteinase (3CLpro) structure: basis for design of anti-SARS drugs. 2003. Science. 300: 1763-1767.

2. Benghezal, Mohammed, Geoffrey O. Wasteneys, and David A. Jones. 2000. The C-terminal dilysine motif confers endoplasmic reticulum localization to type I membrane proteins in plants. The Plant Cell 12: 1179-1201.

4. Eichler, R., O. Lenz, T. Strecker, and W. Garten. 2003. Signal peptide of Lassa virus glycoproteins GP-C exhibits an unusual length. FEBS Lett. 538: 203-6.

5. Fouchier, R. A., T. Kuiken, M. Schutten, G. van Amerongen, G. J. van Doornum, B. G. van den Hoogen, M. Peiris, W. Lim, K. Stohr, and A. D. Osterhaus. 2003. Aetiology: Koch's postulates fulfilled for SARS virus. Nature. 423: 240.

7. Goh, P-Y., Y-J. Tan, S. P. Lim, S. G. Lim, Y. H. Tan, and W. J. Hong. 2001. The Hepatitis C Virus Core protein interacts with NS5A and activates its caspase-mediated proteolytic cleavage. Virol. 290: 224-236.

8. Haijema, B. J., H. Volders, and P. J. Rottier. 2003. Switching species tropism: an effective way to manipulate the feline coronavirus genome. Virol. 77: 4528-38.

12. Klumperman, J., J. K. Locker, A. Meijer, M. C. Horzinek, H. J. Geuze, and P. J. Rottier. 1994. Coronavirus M proteins accumulate in the Golgi complex beyond the site of virion budding. J. Virol. 68: 6523-6534.

13. Haagmans, B. L., H. F. Egberink, and M. C. Horzinek. 1996. Apoptosis and T-cell depletion during feline infectious peritonitis. J. Virol. 70:8977-8983.

16. Marra, M. A., S. J. Jones, C. R. Astell, R. A. Holt, A. Brooks-Wilson, Y. S. Butterfield, J. Khattra, J. K. Asano, S. A. Barber, S. Y. Chan, A. Cloutier, S. M. Coughlin, D. Freeman, N. Girn, O. L. Griffith, S. R. Leach, M. Mayo, H. McDonald, S. B. Montgomery, P. K. Pandoh, A. S. Petrescu, A. G. Robertson, J. E. Schein, A. Siddiqui, D. E. Smailus, J. M. Stott, G. S. Yang, F. Plummer, A. Andonov, H. Artsob, N. Bastien, K. Bernard, T. F. Booth, D. Bowness, M. Czub, M. Drebot, L. Fernando, R. Flick, M. Garbutt, M. Gray, A. Grolla, S. Jones, H. Feldmann, A. Meyers, A. Kabani, Y. Li, S. Normand, U. Stroher, G. A. Tipples, S. Tyler, R. Vogrig, D. Ward, B. Watson, R. C. Brunham, M. Krajden, M. Petric, D. M. Skowronski, C. Upton, and R. L. Roper. 2003. The Genome sequence of the SARS-associated coronavirus. Science. 300: 1399-1404.

17. Nguyen, V. P. and B. G. Hogue. 1997. Protein interactions during coronavirus assembly. J. Virol. 71: 9278-9284.

18. Lai, M. M. C., and D. Cavanagh. 1997. The molecular biology of coronaviruses. Adv. Virus Res. 48:1-100.

19. Pelham, H. R. B., K. G. Hardwick, and M. J. Lewis. 1988. Sorting of soluble ER proteins in yeast. EMBO J. 7: 1757-1762.

20. Rota, P. A., Oberste M S, Monroe S S, Nix W A, Campagnoli R, Icenogle J P, Penaranda S, Bankamp B, Maher K, Chen M H, Tong S, Tamin A, Lowe L, Frace M, DeRisi J L, Chen Q, Wang D, Erdman D D, Peret T C, Burns C, Ksiazek T G, Rollin P E, Sanchez A, Liffick S, Holloway B, Limor J, McCaustland K, Olsen-Rasmussen M, Fouchier R, Gunther S, Osterhaus, A. D., C. Drosten, M. A. Pallansch, L. J. Anderson, and W. J. Bellini. 2003. Characterization of a novel coronavirus associated with severe acute respiratory syndrome. Science. 300: 1394-1399.

21. Ruan, YiJun, Chia Lin Wei, Ling Ai Ee, Vinsensius B. Vega, Herve Thoreau, Se Thoe Su Yun, Jer-Ming Chia, Patrick Ng, Kuo Ping Chiu, Landri Lim, Zhang Tao, Chan Kwai Peng, Lynette Oon Lin Ean, Ng Mah Lee, Leo Yee Sin, Lisa F. P. Ng, Ren Ee Chee, Lawrence W. Stanton, Philip M. Long, and Edison T. Liu. 2003. Comparative full-length genome sequence analysis of 14 SARS coronavirus isolates and common mutations associated with putative origins of infection. Lancet. 361: 1779-85.

22. Nishioka, W. K., and R. M. Welsh. 1993. B cells induce apoptosis via a novel mechanism in fibroblasts infected with mouse hepatitis virus. Nat. Immunol. 12:113-127.

23. Adams J M, Cory S (2001) Life-or-death decisions by the Bcl-2 protein family. Trends Biochem Sci 26:61-66

24. Boise L H, Gonzalez-Garcia M, Postema C E, Ding L, Lindsten T, Turka L A, Mao X, Nunez G, Thompson C B (1993) bcl-x, a bcl-2-related gene that functions as a dominant regulator of apoptotic cell death. Cell 74:597-608

25. Gross A, McDonnell J M, Korsmeyer S J (1999) BCL-2 family members and the mitochondria in apoptosis. Genes Dev 13:1899-1911

26. Tan, Y. -J., S. P. Lim, A. E. Ting, P. -Y. Goh, Y. H. Tan, S. G. Lim, and W. Hong. 2003. An anti-HIV-1 gp120 antibody expressed as an endocytotic transmembrane protein mediates internalization of HIV-1. Virology 315:80-92.

27. Tan, Y. -J., B. C. Fielding, P. -Y. Goh, S. Shen, T. H. P. Tan, S. G. Lim, and W. Hong. 2004a. Over-expression of 7a, a protein specifically encoded by the severe acute respiratory syndrome (SARS)-coronavirus, induces apoptosis via a caspase-dependent pathway. J. Virol. 78:14043-14047.

28. Tsujimoto Y, Cossmann J, Jaffe E, Croce C M (1985) Involvement of the bcl-2 gene in human follicular lymphoma. Science 228:1440-1443

29. Tsujimoto Y, Shimizu S (2000) Bcl-2 family: life-or-death switch. FEBS Lett 466:6-10

30. Vander Heiden M G, Thompson C B (1999) Bcl-2 proteins: regulators of apoptosis or of mitochondrial homestaiss? Nat Cell Biol 1:E209-216

31. Winnefeld, M., Rommelaere J. and C. Cziepluch, 2004. The human small glutamine-rich TPR-containing protein is required for progress through cell division. Experimental Cell Research. Volume 293, Issue 1. 43-57.

32. Wu, S. J., Liu F. H., Hu S. M. and Wang C. 2001. Different combinations of the heat-shock cognate protein 70 (hsc70) C-terminal functional groups are utilized to interact with distinct tetratricopeptide repeat-containing proteins. Biochem J. 15; 359 (Pt 2). 59-26.

33. Schantl J A, Roza M, De Jong A P, Strous G J. 2003. Small glutamine-rich tetratricopeptide repeat-containing protein (hSGT) interacts with the ubiquitin-dependent endocytosis (UbE) motif of the growth hormone receptor. Biochem J. 1; 373 (Pt 3). 855-63.

34. Angeletti P C, Walker D, Panganiban A T. 2002. Small glutamine-rich protein/viral protein Ubinding protein is a novel cochaperone that affects heat shock protein 70 activity. Cell Stress Chaperones. 7 (3). 258-268.

35. Wu S J, Liu F H, Hu S M, Wang C. 2001. Different combinations of the heat-shock cognate protein 70 (hsc70) C-terminal functional groups are utilized to interact with distinct tetratricopeptide repeat-containing proteins. Biochem J.15; 359. (Pt 2). ˜37.59-426.

36. Tan, Yee-Joo, Phuay-Yee Goh, Burtram C. Fielding, Shuo Shen, Chih-Fong Chou, Jian-Lin Fu, Hoe Nam Leong, Yee Sin Leo, Eng Eong Ooi, Ai Ee Ling, Seng Gee Lim, and Wanjin Hong. 2004. Profile of antibody responses against SARS-Coronavirus recombinant proteins and their potential use as diagnostic markers. Clin. Diagn. Lab. Immunol. 11 (2). 362-371.

37. Thiel, V., K. A Ivanov, A. Putics, T. Hertzig, B. Schelle, S. Bayer, B. Weissbrich, E. J. Snijder, H. Rabenau, H. W. Doerr, A. E. Gorbalenya, and J. Ziebuhr. 2003. Mechanisms and enzymes involved in SARS coronavirus genome expression. J. Gen. Virol. 84: 2305-2315.

38. Burtram C. Fielding, Yee-Joo Tan, Shen Shuo, Timothy Tan, Eng-Eong Ooi, Seng Gee Lim, Wanjin Hong, and Phuay-Yee Goh. 2004. Characterization of a unique group-specific protein (U122) from the Severe Acute Respiratory Syndrome (SARS) coronavirus. J Virol. 78 (14). 7311-8.

39. Snijder, E. J., P. J. Bredenbeek, J. C. Dobbe, V. Thiel, J. Ziebuhr, L. L. Poon, Y. Guan, M. Rozanov, W. J. Spaan, and A. E. Gorbalenya. 2003. Unique and conserved features of genome and proteome of SARS-coronavirus, an early split-off from the coronavirus group 2 lineage. J. Mol. Biol. 331: 991-1004.

40. Kuiken, T., R. A. Fouchier, M. Schutten, G. F. Rimmelzwaan, G. van Amerongen, D. van Riel, J. D. Laman, T. de Jong, G. van Doornum, W. Lim, A. E. Ling, P. K. Chan, J. S. Tam, M. C. Zambon, R. Gopal, C. Drosten, S. van der Werf, N. Escriou, J. C. Manuguerra, K. Stohr, J. S. Peiris, and A. D. Osterhaus. 2003. Newly discovered coronavirus as the primary cause of severe acute respiratory syndrome. Lancet. 362: 263-270.

41. Wang, H., Q. Zhang and D. Zhu. 2003. hSGT interacts with the N-terminal region of myostatin. BBRC. 311. 877-883.

42. Shen, S., Z. L. Wen, and D. X. Liu. 2003. Emergence of a coronavirus infectious bronchitis virus mutant with a truncated 3b gene: functional characterization of the 3b protein in pathogenesis and replication. Virol. 311: 16-27.

43. Ortego J, Sola I, Almazan F, Ceriani J E, Riquelme C, Balasch M, Plana J, Enjuanes L. 2003. Transmissible gastroenteritis coronavirus gene 7 is not essential but influences in vivo virus replication and virulence. Virol. 308 (1): 13-22.

44. Haijema B J, Volders H, Rottier P J. 2004. Live, attenuated coronavirus vaccines through the directed deletion of group-specific genes provide protection against feline infectious peritonitis. J Virol. 78 (8): 3863-3871.

45. Callahan M A, Handley M A, Lee Y H, Talbot K J, Harper J W, Panganiban A T. 1998. Functional interaction of human immunodeficiency virus type 1 Vpu and Gag with a novel member of the tetratricopeptide repeat protein family. J Virol. 72 (6): 5189-5197. Erratum in: J Virol. 1998; 72 (10): 8461.

46. Blatch G L, Lassle M. 1999. Review: The tetratricopeptide repeat: a structural motif mediating protein-protein interactions. Bioessays. 21 (11): 932-939.

47. Shen S, Lin P S, Chao Y C, Zhang A, Yang X, Lim S G, Hong W and Tan Y J. 2005. The severe acute respiratory syndrome coronavirus 3a is a novel structural protein. BBRC. 330 (1):286-292.

48. Tan, Y. -J., E. Teng, S. Shen, T. H. P. Tan, P. -Y. Goh, B. C. Fielding, E. -E. Ooi, H. -C. Tan, S. G. Lim, and W. Hong. 2004b. A novel SARS coronavirus protein, U274, is transported to the cell surface and undergoes endocytosis. J. Virol. 78:6723-6734.

49. Nicholas, Karl B. and Nicholas Hugh B, Jr. 1997. Genedoc: a tool for editing and annotating multiple sequence alignments. Distributed by author: psc.edu/biomed/genedoc.

50. Cziepluch C, Kordes E, Poirey R, Grewenig A, Rommelaere J, Jauniaux J C. Identification of a novel cellular TPR-containing protein, SGT, that interacts with the nonstructural protein NS1 of parvovirus H-1. J Virol. 1998 May; 72(5):4149-56.

That which is claimed:

1. An isolated expressed SARS coronavirus group-specific polypeptide 7a.

2. The SARS coronavirus group-specific polypeptide 7a. of claim 1 comprising the amino acid sequence set forth in SEQ ID NO: 2.

3. A vector comprising a nucleic acid sequence encoding the SARS coronavirus group-specific polypeptide of claim 1.

4. An isolated ligand that selectively binds to the SARS coronavirus group-specific polypeptide of claim 1.

5. The ligand of claim 4, wherein said ligand is an antibody or functional fragment thereof.

6. A method of identifying a compound that interacts with the SARS coronavirus group-specific polypeptide of claim 1, the method comprising the steps of:
(a) contacting the SARS coronavirus group-specific polypeptide with a candidate compound under conditions suitable to permit interaction of the candidate compound to the polypeptide; and
(b) detecting the interaction between said candidate compound and the polypeptide.

7. The method of claim 6, wherein said interaction is detected by adding a labelled substrate and measuring a change in the labelled substrate.

8. A method of identifying a compound that binds to the SARS coronavirus group-specific polypeptide of claim 1 comprising the steps of:
(a) contacting the SARS coronavirus group-specific polypeptide with a candidate compound; and
(b) assaying for the formation of a complex between the candidate compound and the polypeptide.

9. The method of claim 8, wherein said assay is selected from the group consisting of a competitive binding assay, a two-hybrid assay and an immunoprecipitation assay.

10. A method of screening for a compound that modulates the activity of the SARS coronavirus group-specific polypeptide of claim 1 comprising the steps of:
(a) contacting the SARS coronavirus group-specific polypeptide with a candidate compound under conditions suitable to enable interaction of the candidate compound to the polypeptide; and
(b) assaying for activity of the polypeptide.

11. The method of claim 10, wherein said modulation comprise an inhibition of activity of the SARS coronavirus group-specific polypeptide.

12. The method of claim 11, wherein said inhibition in activity of the SARS coronavirus group-specific polypeptide comprises inhibiting the formation of a complex between the SARS coronavirus group-specific polypeptide of claim 1 and one or more SARS coronavirus group-specific polypeptides.

13. A method of identifying an agent which is an inhibitor of infection by a SARS coronavirus, the method comprising contacting a cell or cell extract with one or more candidate agents, determining whether there is a decrease in the activity of the SARS coronavirus group-specific polypeptide of claim 1, and thereby determining whether the agent is an inhibitor of a SARS coronavirus.

14. A method of identifying an agent suitable for use in the treatment or prevention of SARS coronavirus in a subject, the method comprising:
(a) obtaining a biological sample from the subject,
(b) contacting the sample with a candidate agent,
(c) determining whether there is a decrease in the activity of the polypeptide of claim 1, and
thereby determining whether the agent is suitable for use in the treatment of SARS coronavirus.

15. A method for treating or preventing SARS coronavirus in a subject, the method comprising administering to the subject a therapeutically effective amount of the ligand of claim 4.

16. The method of claim 15, wherein said method comprises inhibition of apoptosis associated with the overexpression of the polypeptide of claim 1.

17. The method of claim 16, wherein inhibition of apoptosis comprises inhibiting the formation of a complex between the SARS coronavirus group-specific polypeptide of claim 1 and one or more SARS coronavirus group-specific polypeptides.

18. A method of diagnosing SARS coronavirus infection in a subject, the method comprising the steps of:
(a) obtaining a biological sample from the subject; and
(b) assaying for expression of the SARS coronavirus group-specific polypeptide of claim 1.

19. The method of claim 18, wherein the assay comprises contacting the biological sample with an antibody which selectively binds to the SARS coronavirus group-specific polypeptide.

20. A kit comprising the SARS coronavirus group-specific polypeptide of claim 1, together with a diluent or carrier.

21. A method for screening a subject for infection by a SARS coronavirus, the method comprising:
(a) obtaining a biological sample from said subject;
(b) contacting said sample with the ligand of claim 4, and
(c) detecting the presence of the ligand selectively bound to the SARS coronavirus polypeptide of claim 1.

22. The method of claim 21, wherein the biological sample is a plasma or cell sample.

23. A method for screening a subject for infection by a SARS coronavirus, the method comprising:
(a) obtaining a biological sample from said subject;
(b) contacting said biological sample from said subject with the nucleic acid encoding the polypeptide of claim 1; and
(c) detecting the presence or absence of hybridisation between the nucleic acid of said biological sample and the nucleic acid sequence encoding the polypeptide of claim 1.

24. The method of claim 23, wherein the nucleic acid sequence corresponds to SEQ ID NO: 1 or a region capable of selectively hybridising to the nucleic acid encoding the SARS coronavirus group-specific polypeptide.

25. The method of claim 24, wherein the region corresponds to the oligonucleotides selected from the group consisting of SEQ ID NOS: 3 to 16.

26. A vaccine, wherein said vaccine comprises the polypeptide of claim 1 together with a pharmaceutically acceptable carrier, adjuvant and/or diluent.

27. The vaccine of claim 26, wherein said vaccine is formulated for administration via an oral, inhalation or parenteral route.

28. A method for inducing an immune response in a subject against infection by a SARS coronavirus, comprising administering to said vertebrate an immunologically effective amount of the polypeptide of claim 1.

29. A method for the treatment and/or prophylaxis of infection by a SARS coronavirus, wherein said method comprises administering a therapeutically effective amount of the vaccine of claim 26.

30. The method of claim 26, wherein said treatment and/or prophylaxis of infection by a SARS coronavirus comprises inhibition of apoptosis associated with the overexpression of the polypeptide of claim 1.

31. The method of claim 30, wherein inhibition of apoptosis comprises inhibiting the formation of a complex between the SARS coronavirus group-specific polypeptide of claim 1 and one or more SARS coronavirus group-specific polypeptides

32. A method for the treatment and/or prophylaxis of cancer, wherein said method comprises administering to a subject a therapeutically effective amount of the SARS coronavirus group-specific polypeptide of claim 1.

33. The method of claim 32, wherein said treatment and/or prophylaxis of infection by a SARS coronavirus comprises promotion of apoptosis associated with the overexpression of the polypeptide of claim 1.

34. A host protein capable of interaction with the SARS coronavirus group-specific polypeptide of claim 1.

35. The host protein of claim 34, wherein said protein comprises small glutamine-rich tetratricopeptide (SGT) repeat-containing protein.

36. The host protein of claim 35, wherein said small glutamine-rich tetratricopeptide (SGT) repeat-containing protein is degraded by the SARS coronavirus group-specific polypeptide.

37. An agent for inhibiting apoptosis by SARS corona virus group-specific polypeptide of either claim 1 comprising one or more proteins from the family of Bcl-2 related proteins.

38. The agent according to claim 37, wherein the one or more proteins are selected from the group consisting of BC1-X_{L}, BAD or combinations thereof.

39. A method for the treatment and/or prophylaxis of infection by a SARS coronavirus, wherein said method comprises administering to a subject a therapeutically effective amount of the agent of either claim 37.

40. The method according to claim 39, wherein said treatment and/or prophylaxis of infection by a SARS coronavirus comprises inhibition of apoptosis associated with the overexpression of the polypeptide of either claim 1.

41. The method according to claim 40, wherein the inhibition of apoptosis comprises inhibiting the formation of a complex between the SARS coronavirus group-specific polypeptide of either claim 1 and one or more SARS coronavirus group-specific polypeptides.

42. An isolated expressed SARS coronavirus group-specific polypeptide 3b.

43. The SARS coronavirus group-specific polypeptide 3b of claim 42 comprising the amino acid sequence as set forth in SEQ ID NO: 18.

44. A method of identifying an agent which is an inhibitor of infection by a SARS coronavirus, the method comprising contacting a cell or cell extract with one or more candidate agents, determining whether there is a decrease in the activity of the SARS coronavirus group-specific polypeptide of claim 42, and thereby determining whether the agent is an inhibitor of a SARS coronavirus.

45. A method of identifying an agent suitable for use in the treatment or prevention of SARS coronavirus in a subject, the method comprising:
(a) obtaining a biological sample from the subject,
(b) contacting the sample with a candidate agent,
(c) determining whether there is a decrease in the activity of the polypeptide of claim 42, and
thereby determining whether the agent is suitable for use in the treatment of SARS coronavirus.

46. A method for treating or preventing SARS coronavirus in a subject, the method comprising administering to the subject a therapeutically effective amount of a compound identified by the method of claim 42.

47. The method of claim 46, wherein said method comprises inhibition of cell death associated with the overexpression of the polypeptide of claim 42.

48. The method of claim 47, wherein inhibition of cell death comprises inhibiting the formation of a complex between the SARS coronavirus group-specific polypeptide of claim 42 and one or more polypeptides, such as SARS coronavirus group-specific polypeptides.

49. A vaccine, wherein said vaccine comprises the ligand of claim 4, together with a pharmaceutically acceptable carrier, adjuvant and/or diluent.

50. A method for inducing an immune response in a subject against infection by a SARS coronavirus, comprising administering to said vertebrate an immunologically effective amount of the vaccine of claim 26.

51. A method for treating or preventing SARS coronavirus in a subject, the method comprising administering to the subject a therapeutically effective amount of a compound identified by the method of claim 8.

52. A method for treating or preventing SARS coronavirus in a subject, the method comprising administering to the subject a therapeutically effective amount of a compound identified by the method of claim 10.

53. A method for treating or preventing SARS coronavirus in a subject, the method comprising administering to the subject a therapeutically effective amount of a compound identified by the method of claim 13.

54. A method for treating or preventing SARS coronavirus in a subject, the method comprising administering to the subject a therapeutically effective amount of a compound identified by the method of claim 14.

55. A kit comprising the ligand of claim 4, together with a diluent or carrier.

56. A method for inducing an immune response in a subject against infection by a SARS coronavirus, comprising administering to said vertebrate an immunologically effective amount of the vaccine of claim 26.
